(19) United States
(12) Patent Application Publication
(10) Pub. No.: US 2006/0111310 A1
(43) Pub. Date: May 25, 2006

(54) Composition comprising triterpene saponins and compounds with angeloyl functional group, methods for preparing same and uses thereof

(76) Inventors: Pui-Kwong Chan, Sugarland, TX (US); May Sung Mak, Kowloon (HK); Yun Wang, Dunedin (NZ)

Correspondence Address: LAW OFFICES OF ALBERT WAI-KIT CHAN, LLC, WORLD PLAZA, SUITE 604, 141-07 20TH AVENUE, WHITESTONE, NY, 11357, US

(21) Appl. No.: 11/267,523

(22) Filed: Nov. 4, 2005

Related U.S. Application Data
(63) Continuation-in-part of application No. 11/131,551, filed on May 17, 2005.
(63) Continuation-in-part of application No. 11/117,760, filed on Apr. 27, 2005.
(63) Continuation-in-part of application No. 10/906,303, filed on Feb. 14, 2005.
(63) Continuation-in-part of application No. PCT/US04/43465, filed on Dec. 23, 2004.
Application No. PCT/US04/43465 is a continuation-in-part of application No. PCT/US04/33359, filed on Oct. 8, 2004.
(63) Continuation-in-part of application No. PCT/US05/31900, filed on Sep. 7, 2005.
(60) Provisional application No. 60/532,101, filed on Dec. 23, 2003.
(60) Provisional application No. 60/509,851, filed on Oct. 9, 2003.
(60) Provisional application No. 60/617,379, filed on Oct. 8, 2004.
(60) Provisional application No. 60/613,811, filed on Sep. 27, 2004.
(60) Provisional application No. 60/607,858, filed on Sep. 7, 2004.
(60) Provisional application No. 60/675,282, filed on Apr. 27, 2005.
(60) Provisional application No. 60/675,284, filed on Apr. 27, 2005.

(51) Int. Cl. C07H 15/24; A61K 31/704

(52) U.S. Cl. 514/33; 536/18.1

(57) ABSTRACT

This invention provides a compound comprising a triterpenoidal saponin, triterpenoid, triterpenoidal compound or sapongenin, comprising at least two side groups selected from the group consisting of: angeloyl groups, tigloyl groups and senecioyl groups, wherein the side groups are attached to carbon 21, 22 or/and 28 of triterpenoidal saponin, triterpenoid, triterpenoidal compound or sapongenin backbone. This invention provides a composition for inhibiting tumor cell growth, comprising an appropriate amount of a triterpenoidal saponin, triterpenoid, triterpenoidal compound or sapongenin, wherein the triterpenoidal saponin, triterpenoid, triterpenoidal compound or sapongenin comprises any two side groups selected from the group consisting of: angeloyl groups, tigloyl groups and senecioyl groups, wherein the side groups are attached to carbon 21, 22 or/and 28 of triterpenoidal saponin, triterpenoid, triterpenoidal compound or sapongenin backbone.

This application is a Continuation-In-Part of International Application No. PCT/US05/31900, filed Sep. 7, 2005, Continuation-In-Part of U.S. Ser. No. 11/131,551, filed May 17, 2005, Continuation-In-Part of U.S. Ser. No. 11/117,760, filed Apr. 27, 2005, Continuation-In-Part of U.S. Ser. No. 10/906,303, filed Feb. 14, 2005, Continuation-In-Part of International Application No. PCT/US04/43465, filed Dec. 23, 2004, which is a Continuation-In-Part of International Application No. PCT/US04/33359, filed Oct. 8, 2004, which claims the benefit of U.S. Ser. Nos. 60/532,101, filed Dec. 23, 2003, and 60/509,851, filed Oct. 9, 2003; and International Application No. PCT/US05/31900, filed Sep. 7, 2005, claims the benefit of U.S. Ser. Nos. 60/617,379, filed Oct. 8, 2004, 60/613,811, filed Sep. 27, 2004, and 60/607,858, filed Sep. 7, 2004, 60/675,282, Filed Apr. 27, 2005, and 60/675,284, Filed Apr. 27, 2005. The contents of these preceding applications are hereby incorporated in their entireties by reference into this application.

Throughout this application, various publications are referenced. Disclosures of these publications in their entireties are hereby incorporated by reference into this application in order to more fully describe the state of the art to which this invention pertains.

### FIELD OF THE INVENTION

This invention relates to saponins, and compounds with angeloyl groups isolated from plants, their uses and functions, and methods of their preparations.

### SUMMARY OF THE INVENTION

In accordance with these and other objects of the invention, a brief summary of the present invention is presented. Some simplifications and omission may be made in the following summary, which is intended to highlight and introduce some aspects of the present invention, but not to limit its scope. Detailed descriptions of a preferred exemplary embodiment adequate to allow those of ordinary skill in the art to make and use the invention concepts will follow in later sections.

This invention provides a compound comprising a triterpenoidal saponin, triterpenoid, triterpenoidal compound or sapongenin, comprising two angeloyl groups, or at least two side groups selected from the group consisting of: angeloyl groups, tigloyl groups and senecioyl groups, wherein the side groups are attached to carbon 21, 22 or/and 28 of triterpenoidal saponin, triterpenoid, triterpenoidal compound or sapongenin backbone.

This invention provides a composition for inhibiting tumor cell growth, comprising an appropriate amount of a triterpenoidal saponin, triterpenoid, triterpenoidal compound or sapongenin, wherein the triterpenoidal saponin, triterpenoid, triterpenoidal compound or sapongenin comprises two angeloyl groups or any two side groups selected from the group consisting of: angeloyl groups, tigloyl groups and senecioyl groups, wherein the side groups are attached to carbon 21 and 22 of triterpenoidal saponin, triterpenoid, triterpenoidal compound or sapongenin backbone. In an embodiment, the side groups are attached to carbon 21, 22 or/and 28 of triterpenoidal saponin, triterpenoid, triterpenoidal compound or sapongenin backbone.

The invention provides the methods and uses of triterpenoidal saponins purified and isolated from plants.

This invention provides compositions comprising the triterpenoidal saponins or their derivatives for inhibition of tumor growth. The compounds comprise angeloyl group(s) or tigloyl group(s) or senecioyl group(s) or combinations thereof which are attached to carbon 21, 22 or/and 28 of their sapongenines. In an embodiment, the compounds may comprise any two angeloyl groups or tigeloyl groups or senecioyl groups or combinations thereof attached to a sugar moiety which bonds to carbon 21 or 22 of their sapongenines. In an embodiment, the side groups are attached to carbon 21, 22 or/and 28 of triterpenoidal saponin, triterpenoid, triterpenoidal compound or sapongenin backbone.

In an embodiment, the saponin comprising a sugar moiety, wherein the sugar moiety comprises at least one sugar, or D-glucose, or D-galactose, or L-rhamnose, or L-arabinose, or D-xylose, or alduronic acid, or D-glucuronic acid or D-galacturonic acid, or their derivative thereof, or the combination thereof.

### DETAILED DESCRIPTION OF THE FIGURES

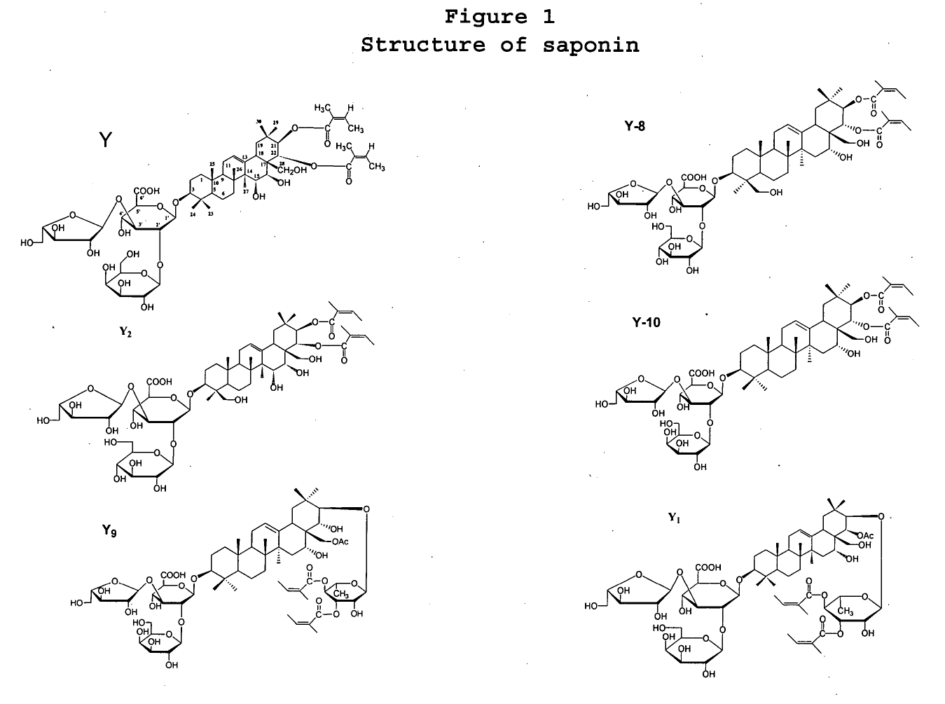

FIG. 1 shows structure of saponins.

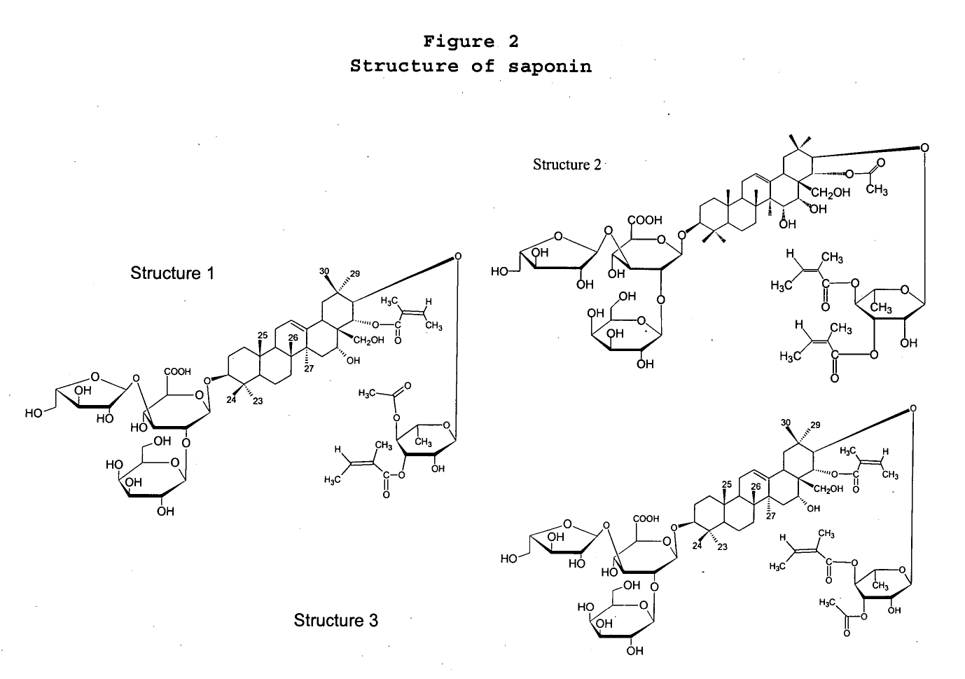

FIG. 2 shows structure of saponins.

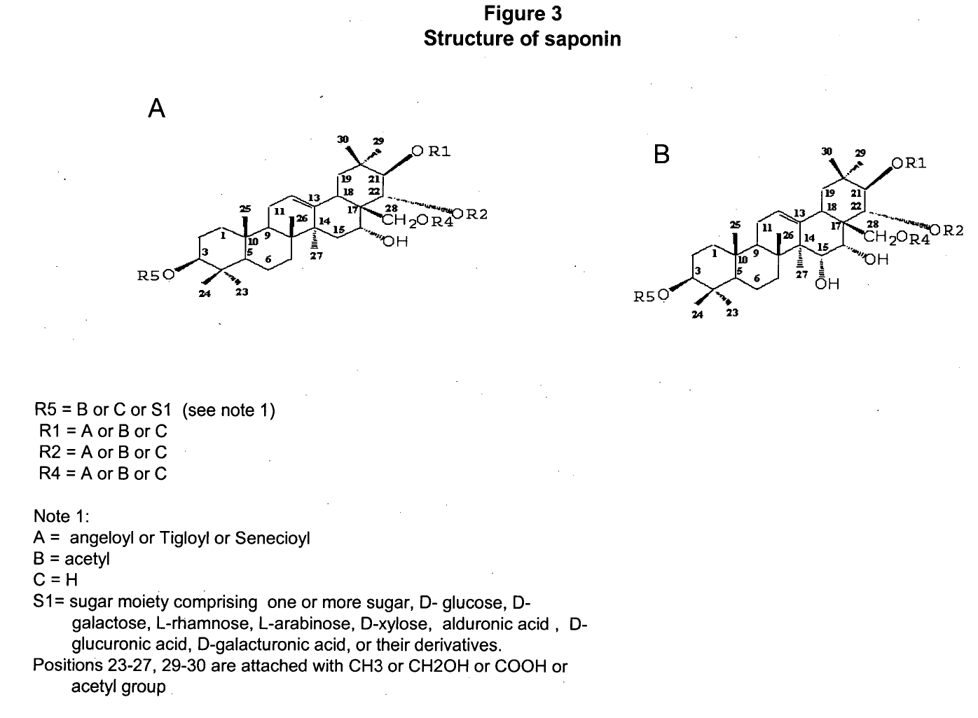

FIG. 3 shows structure of saponins

R5=B or C or S1 (see note 1)

R1=A or B or C

R2=A or B or C

R4=A or B or C

Note 1:

A=angeloyl or Tigloyl or Senecioyl

B=acetyl

C=H

S1=sugar moiety comprising one or more sugar, D-glucose, D-galactose, L-rhamnose, L-arabinose, D-xylose, alduronic acid, D-glucuronic acid, D-galacturonic acid, or their derivatives.

Positions 23-27, 29-30 are attached with CH3 or CH2OH or COOH or acetyl group

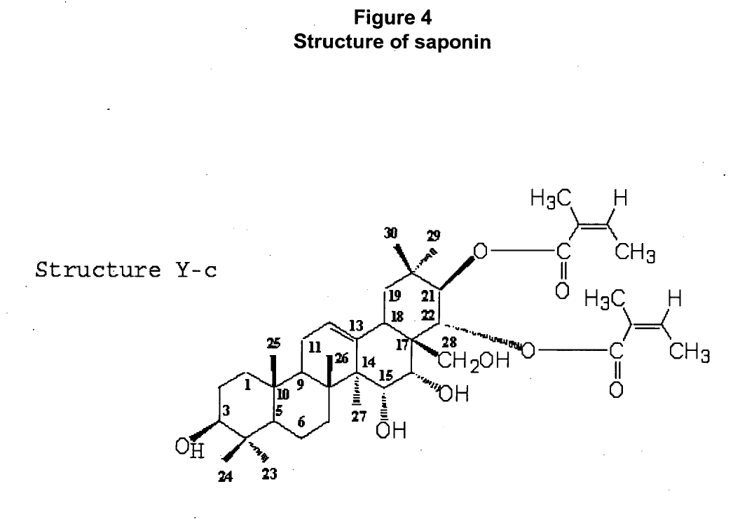

FIG. 4 shows structure of saponins

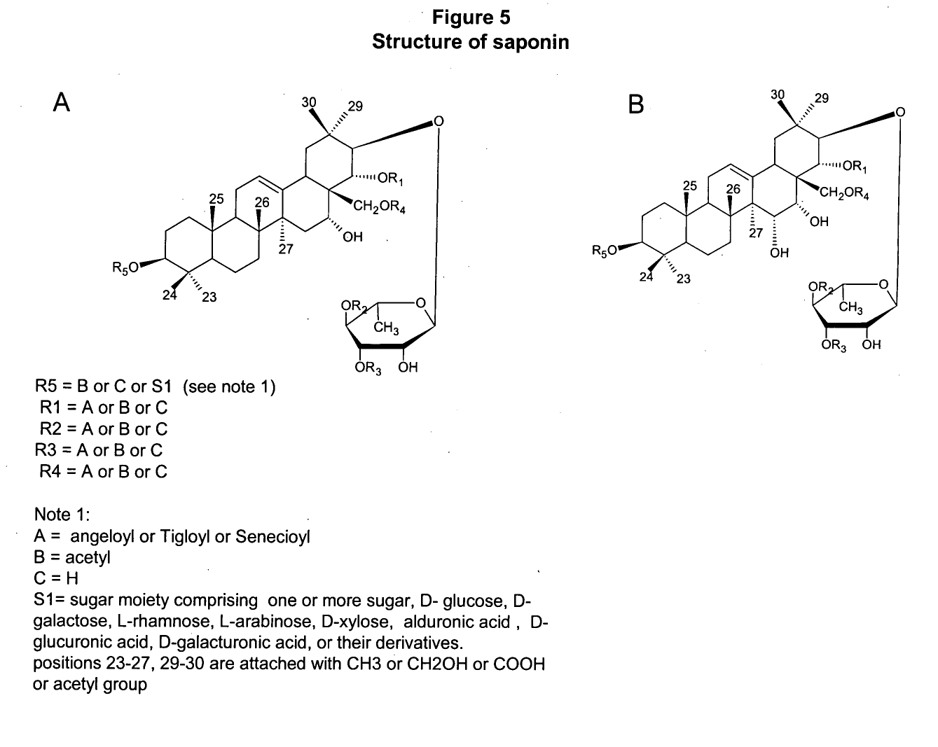

FIG. 5 shows a structure of saponins

R5=B or C or S1 (see note 1)

R1=A or B or C

R2=A or B or C

R3=A or B or C

R4=A or B or C

Note 1:

A=angeloyl or Tigloyl or Senecioyl

B=acetyl

C=H

S1=sugar moiety comprising one or more sugar, D-glucose, D-galactose, L-rhamnose, L-arabinose, D-xylose, alduronic acid, D-glucuronic acid, D-galacturonic acid, or their derivatives.

positions 23-27, 29-30 are attached with CH3 or CH2OH or COOH or acetyl group

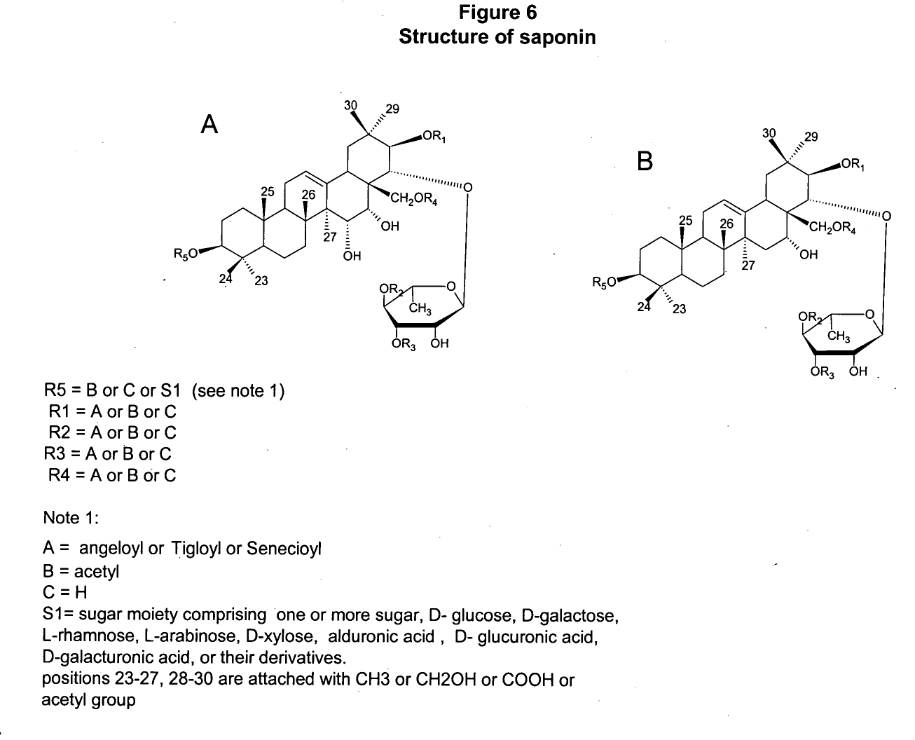

FIG. 6 shows a structure of saponins

R5=B or C or S1 (see note 1)

R1=A or B or C

R2=A or B or C

R3=A or B or C

R4=A or B or C

Note 1:

A=angeloyl or Tigloyl or Senecioyl

B=acetyl

C=H

S1=sugar moiety comprising one or more sugar, D-glucose, D-galactose, L-rhamnose, L-arabinose, D-xylose, alduronic acid, D-glucuronic acid, D-galacturonic acid, or their derivatives.

positions 23-27, 28-30 are attached with CH3 or CH2OH or COOH or acetyl group

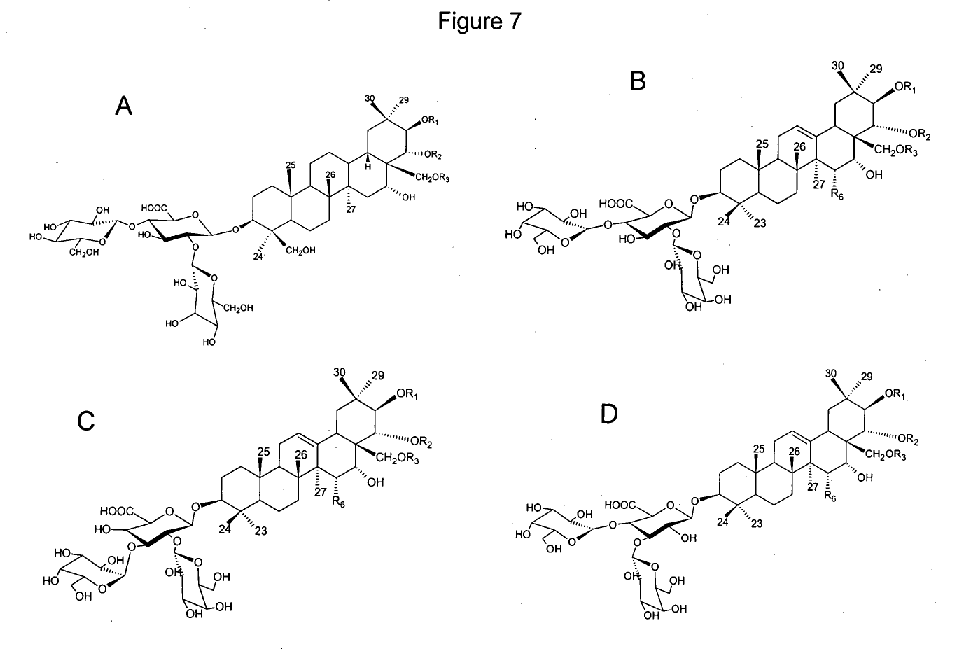

FIG. 7A shows a structure of saponins

Wherein R1=angeloyl group or tigloyl group or senecioyl group or H.

R2=angeloyl group or tigloyl group or senecioyl group or acetyl group or H.

R3=angeloyl group or tigloyl group or senecioyl group or acetyl group or H.

FIG. 7B, C, D shows a structure of saponins

Wherein R1=angeloyl group or tigloyl group or senecioyl group or H.

R2=angeloyl group or tigloyl group or senecioyl group or acetyl group or H.

R3=angeloyl group or tigloyl group or senecioyl group or acetyl group or H.

R6=H or OH

Positiion 23-27 and 28-30 are attached with CH3 or CH2OH or COOH or CHO

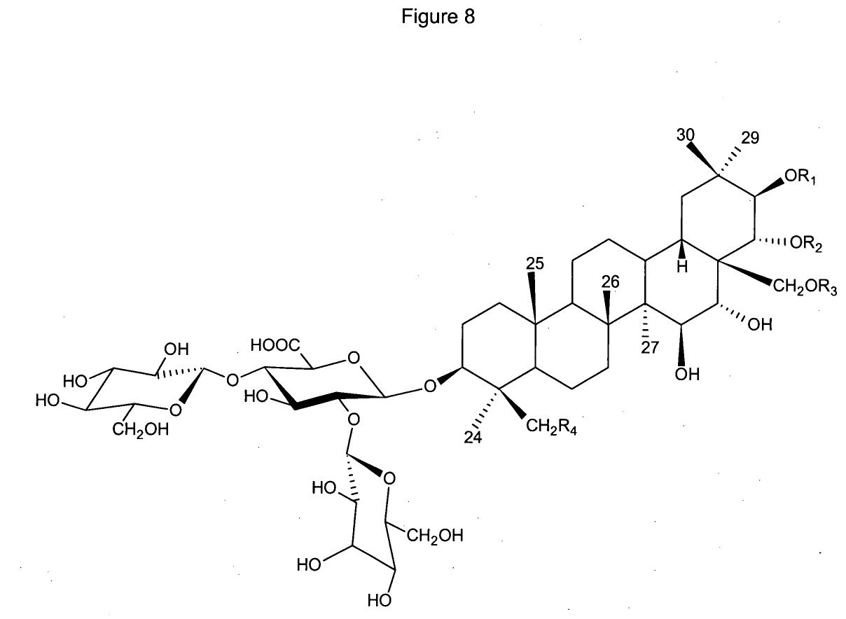

FIG. 8 shows a structure of saponins:

Wherein R1=angeloyl group or tigloyl group or senecioyl group or H.

R2=angeloyl group or tigloyl group or senecioyl group or acetyl group or H.

R3=angeloyl group or tigloyl group or senecioyl group or acetyl group or H.

R4=OH or H

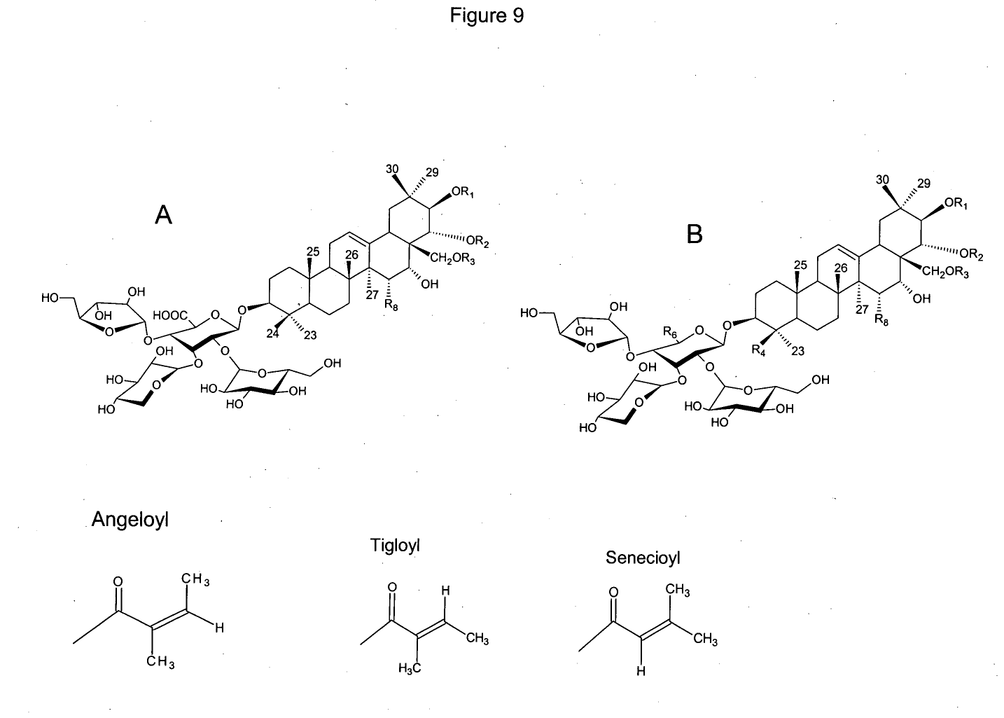

FIG. 9A shows a structure of saponins:

Wherein R1=angeloyl group or tigloyl group or senecioyl group or acetyl group or H.

R2=angeloyl group or tigloyl group or senecioyl group or acetyl group or H.

R3=Acetyl or H.

R8=H or OH

FIG. 9B shows a structure of saponins:

Wherein R1=angeloyl group or tigloyl group or senecioyl group or acetyl group or H.

R2=angeloyl group or tigloyl group or senecioyl group or acetyl group or H.

R3=Acetyl or H.

R4=CH3 or CH2OH or COOH

R6=CH3 or CH2OH or COOH

R8=H or OH

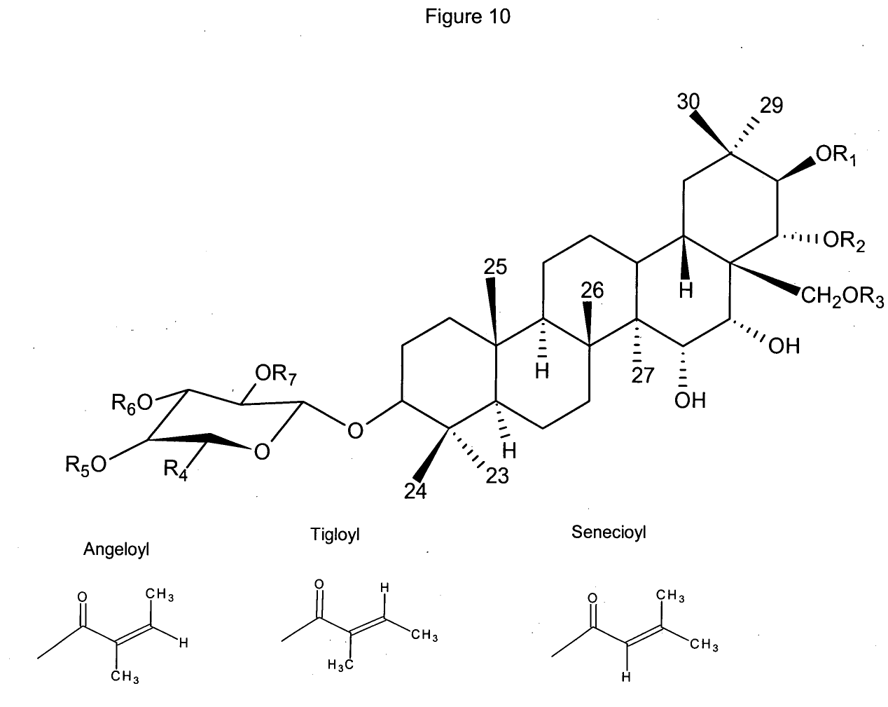

FIG. 10 shows a structure of saponins:

Wherein R1=angeloyl group or tigloyl group or senecioyl group or acetyl group or H.

R2=angeloyl group or tigloyl group or senecioyl group or acetyl group or H.

R3=Acetyl or H.

R4=COOH OR COOMe or CH2OH

R5=α-L-araf and R6=α-L-arap and R7=β-D-glup; or R5, R6, and R7 is a sugar moiety, or D-glucose, or D-galactose, or L-rhamnose, or L-arabinose, or D-xylose, or alduronic acid, or D-glucuronic acid, or D-galacturonic acid, or their derivatives.

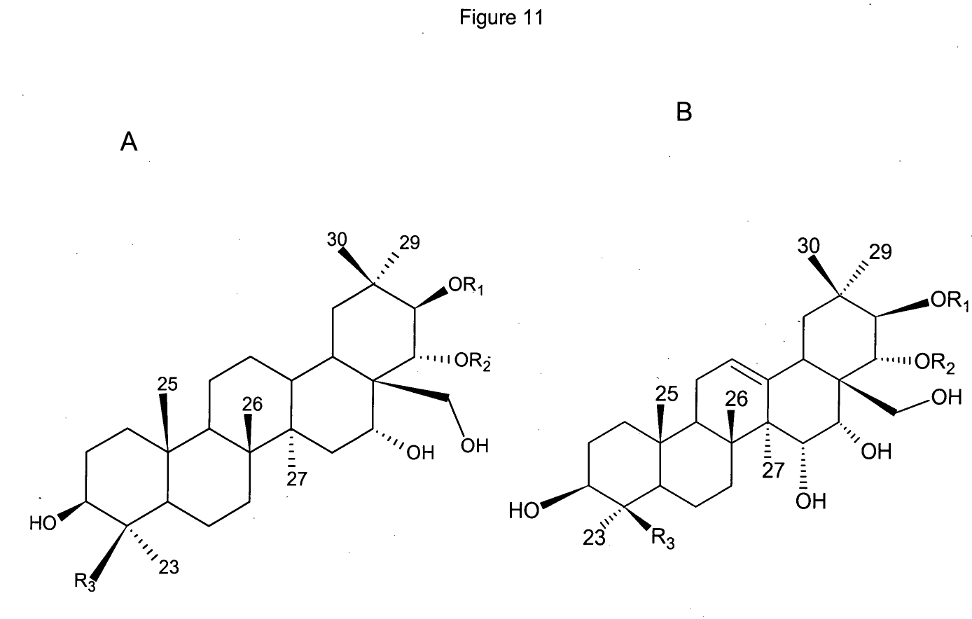

FIG. 11A shows a structure of saponins:

Wherein R1=angeloyl group or tigloyl group or senecioyl group or acetyl group or H.

R2=angeloyl group or tigloyl group or senecioyl group or acetyl group or H.

R3=CH₂OH or CH₃ or CHO

FIG. 11B shows a structure of saponins:

Wherein R1=angeloyl group or tigloyl group or senecioyl group or acetyl group or H.

R2=angeloyl group or tigloyl group or senecioyl group or acetyl group or H.

R3=CH₂OH or CH₃ or CHO

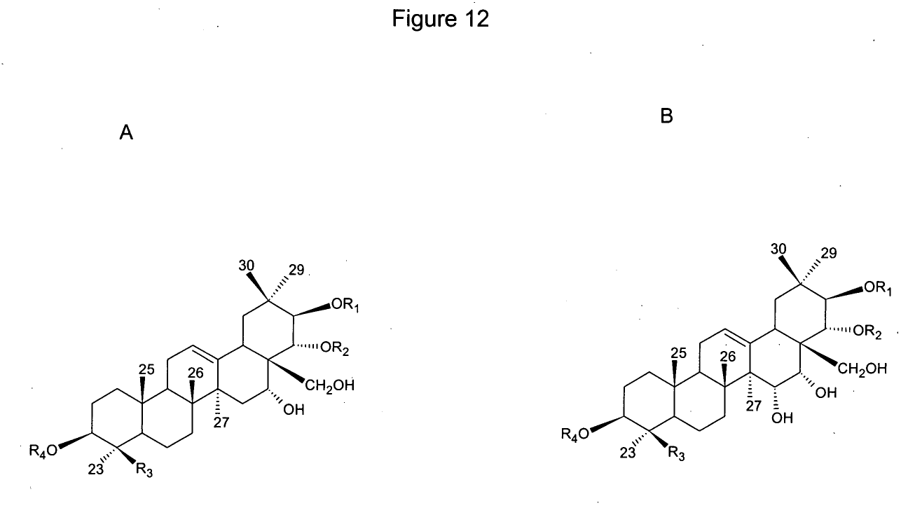

FIG. 12A shows a structure of saponins:

Wherein R1=angeloyl group or tigloyl group or senecioyl group or acetyl group or H.

R2=angeloyl group or tigloyl group or senecioyl group or acetyl group or H.

R3=CH₂OH or CH₃ or CHO or COOCH₃

R4=S1=sugar moiety comprising one or more sugar, D-glucose, D-galactose, L-rhamnose, L-arabinose, D-xylose, alduronic acid, D-glucuronic acid, D-galacturonic acid, or/and their derivatives.

FIG. 12B shows a structure of saponins:

Wherein R1=angeloyl group or tigloyl group or senecioyl group or acetyl group or H.

R2=angeloyl group or tigloyl group or senecioyl group or acetyl group or H.

R3=CH₂OH or CH₃ or CHO or COOCH₃

R4=sugar moiety comprising one or more sugar, D-glucose, D-galactose, L-rhamnose, L-arabinose, D-xylose, alduronic acid, D-glucuronic acid, D-galacturonic acid, or/and their derivatives.

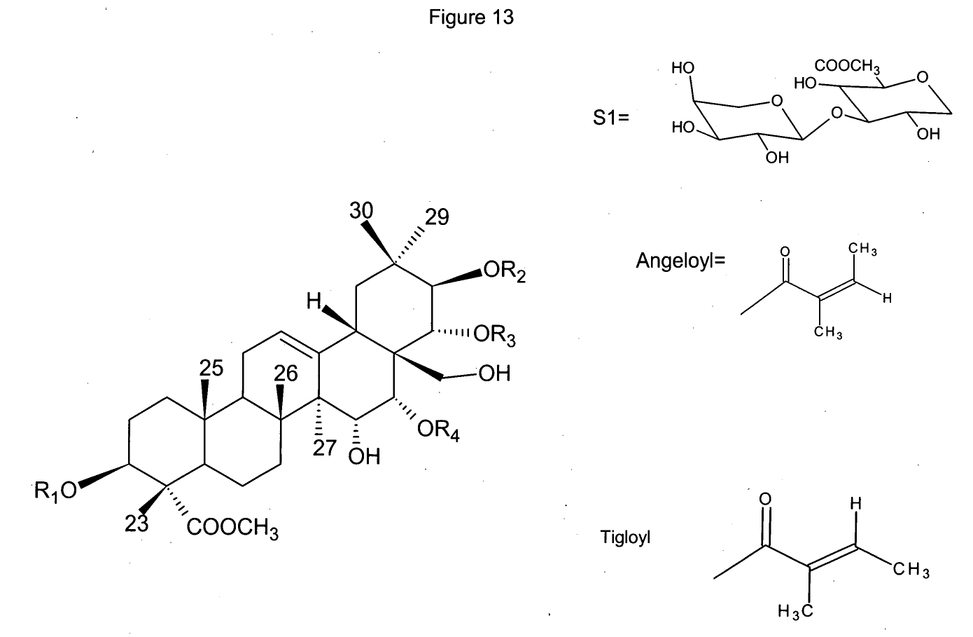

FIG. 13 shows a structure of saponins:

Wherein R2=angeloyl group or tigloyl group or senecioyl group or acetyl group or H.

R3=angeloyl group or tigloyl group or senecioyl group or acetyl group or H.

R4=CH₂OH or CH₃ or CHO or COOCH₃

R1=sugar moiety comprising one or more sugar, D-glucose, D-galactose, L-rhamnose, L-arabinose, D-xylose, alduronic acid, D-glucuronic acid, D-galacturonic acid, or/and their derivatives.

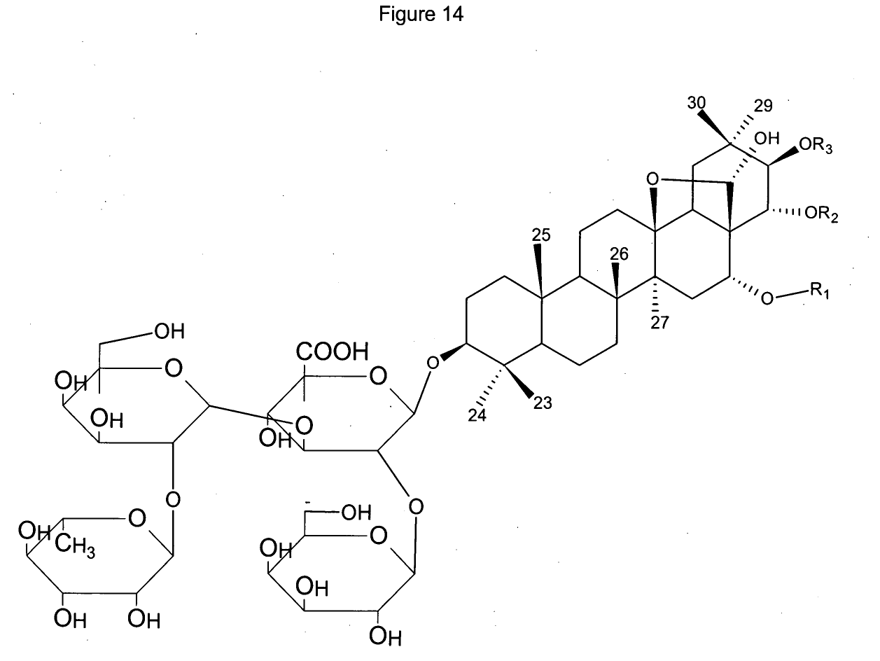

FIG. 14 shows a structure of saponins:

Wherein R1=angeloyl group or tigloyl group or senecioyl group or propanoyl or butanoly or acetyl group or H.

R2=angeloyl group or tigloyl group or senecioyl group or propanoyl or butanoly or acetyl group or H.

R3=angeloyl group or tigloyl group or senecioyl group or propanoyl or butanoly or acetyl group or H.

R4=sugar moiety comprising one or more sugar, D-glucose, D-galactose, L-rhamnose, L-arabinose, D-xylose, alduronic acid, D-glucuronic acid, D-galacturonic acid, or/and their derivatives.

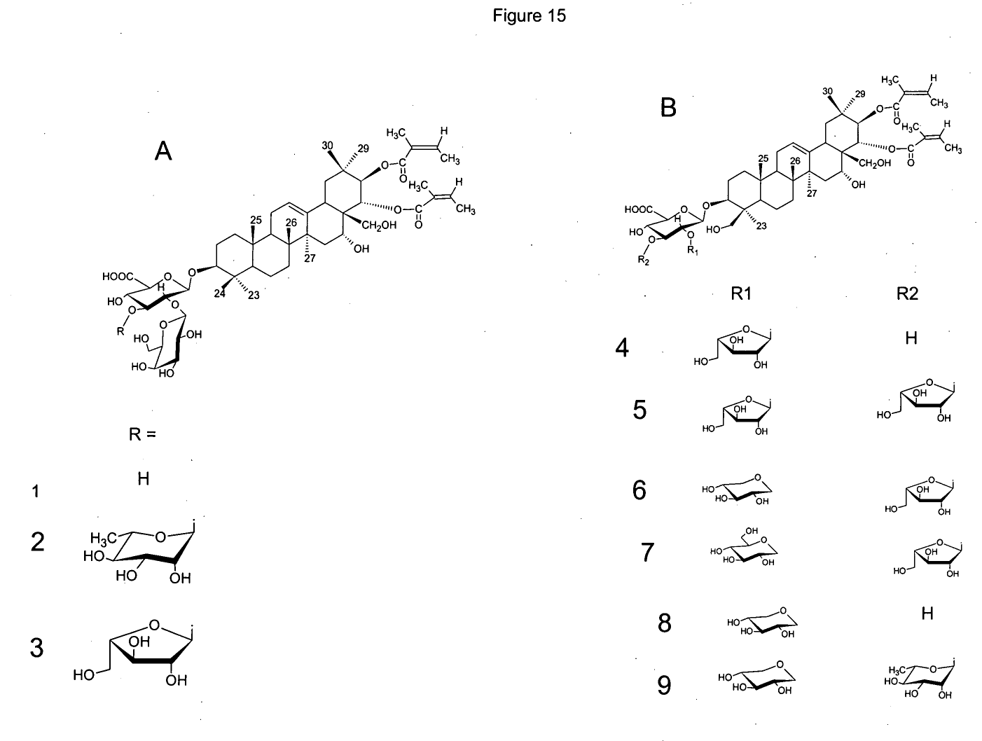

FIG. 15 shows a structure of saponins

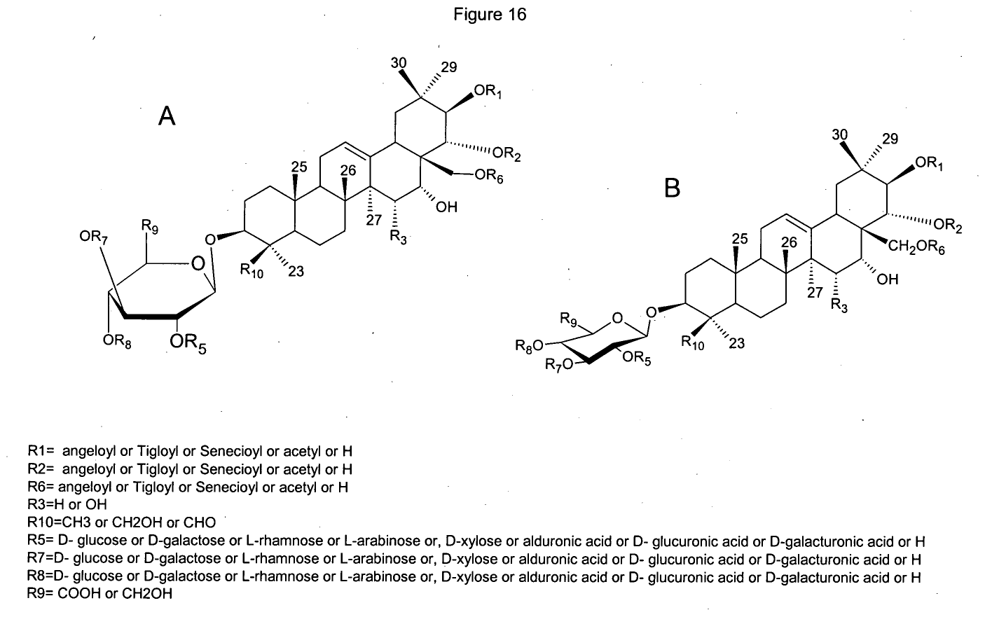

FIG. 16A, 16B shows a structure of saponins

R1=angeloyl or Tigloyl or Senecioyl or acetyl or H

R2=angeloyl or Tigloyl or Senecioyl or acetyl or H

R6=angeloyl or Tigloyl or Senecioyl or acetyl or H

R3=H or OH

R10=CH3 or CH2OH or CHO

R5=D-glucose or D-galactose or L-rhamnose or L-arabinose or, D-xylose or alduronic acid or D-glucuronic acid or D-galacturonic acid or H

R7=D-glucose or D-galactose or L-rhamnose or L-arabinose or, D-xylose or alduronic acid or D-glucuronic acid or D-galacturonic acid or H

R8=D-glucose or D-galactose or L-rhamnose or L-arabinose or, D-xylose or alduronic acid or D-glucuronic acid or D-galacturonic acid or H

R9=COOH or CH₂OH

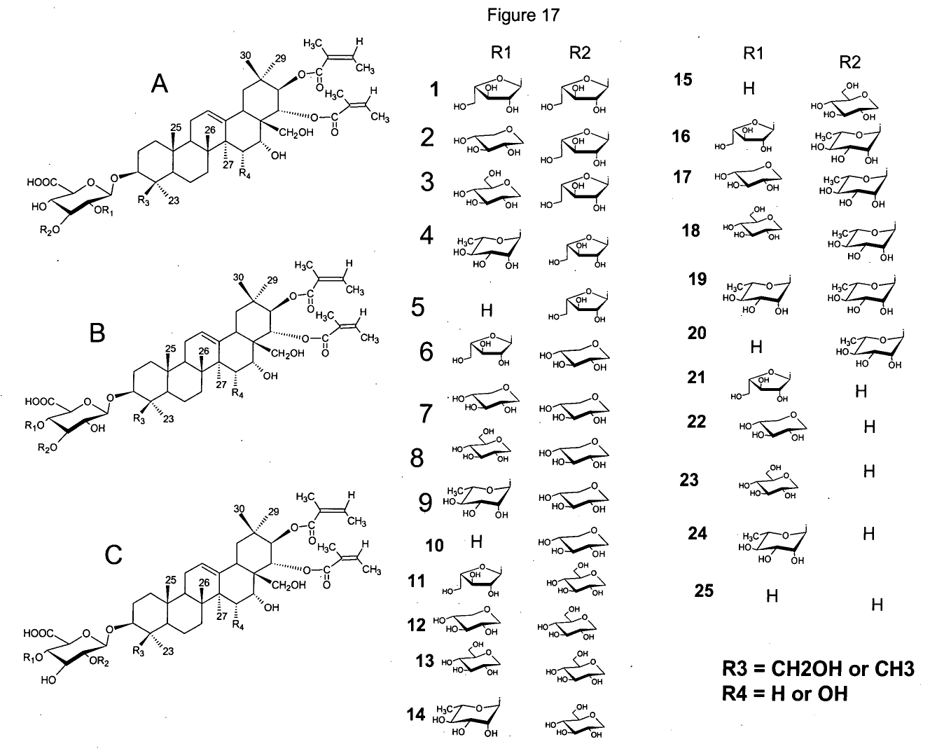

FIG. 17 shows a structure of saponins

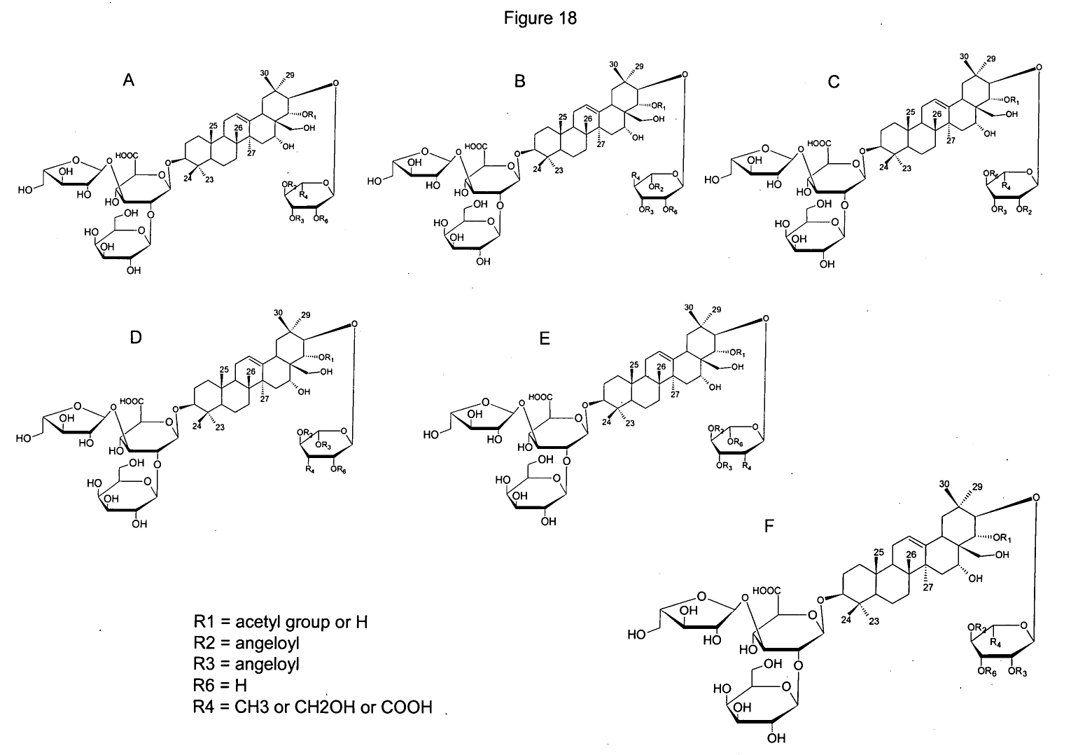

FIG. 18 shows a structure of saponins

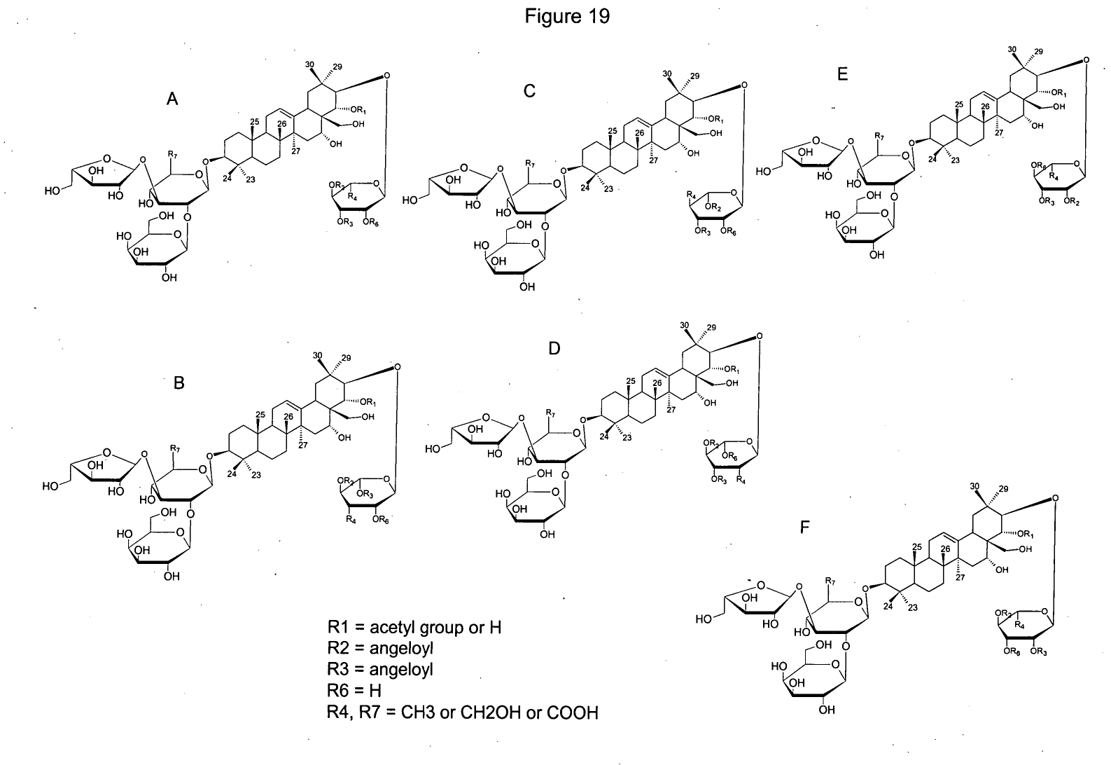

FIG. 19 shows a structure of saponins

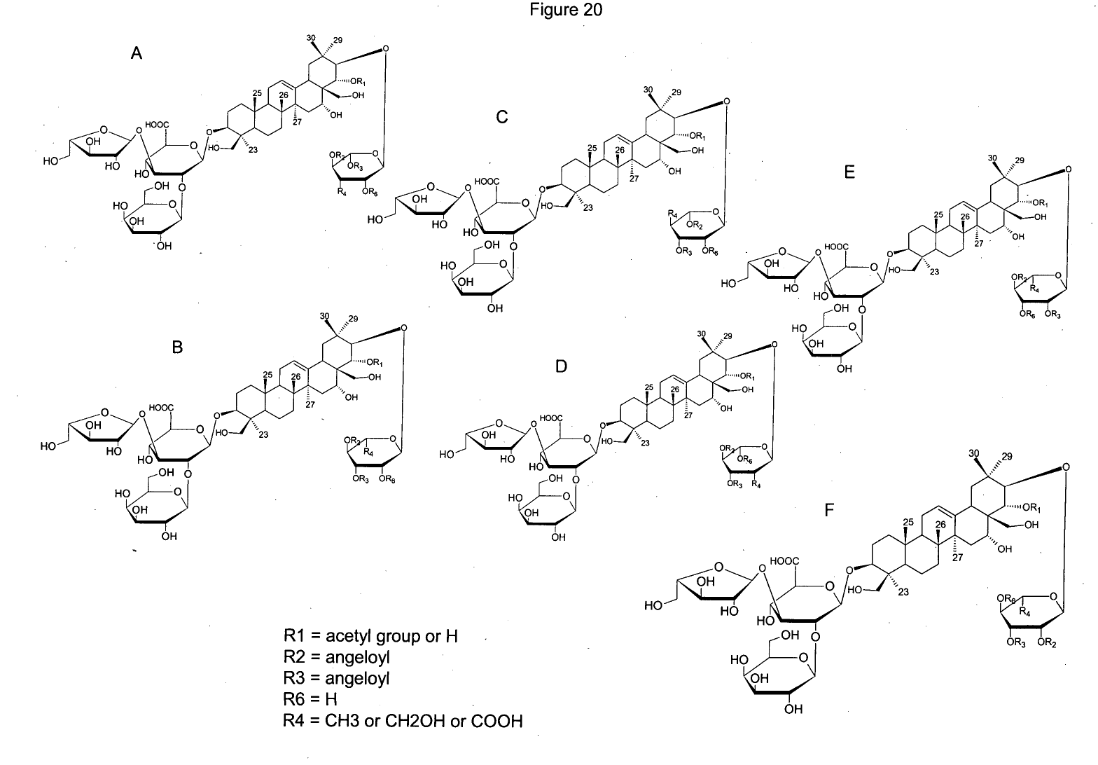

FIG. 20 shows a structure of saponins

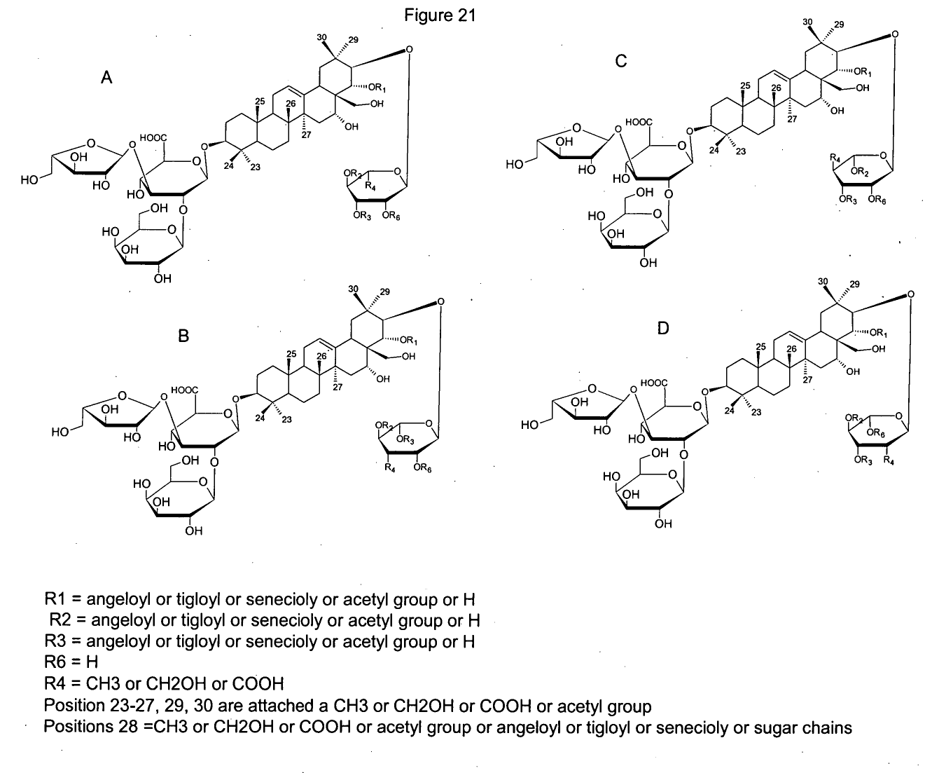

FIG. 21 shows a structure of saponins

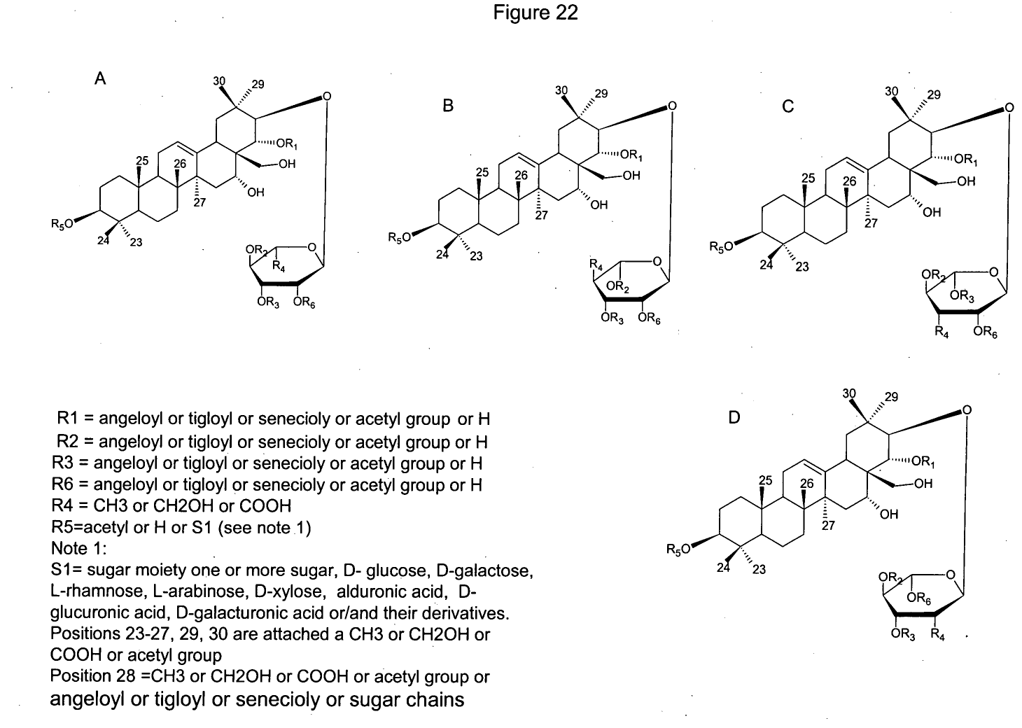

FIG. 22 shows a structure of saponins

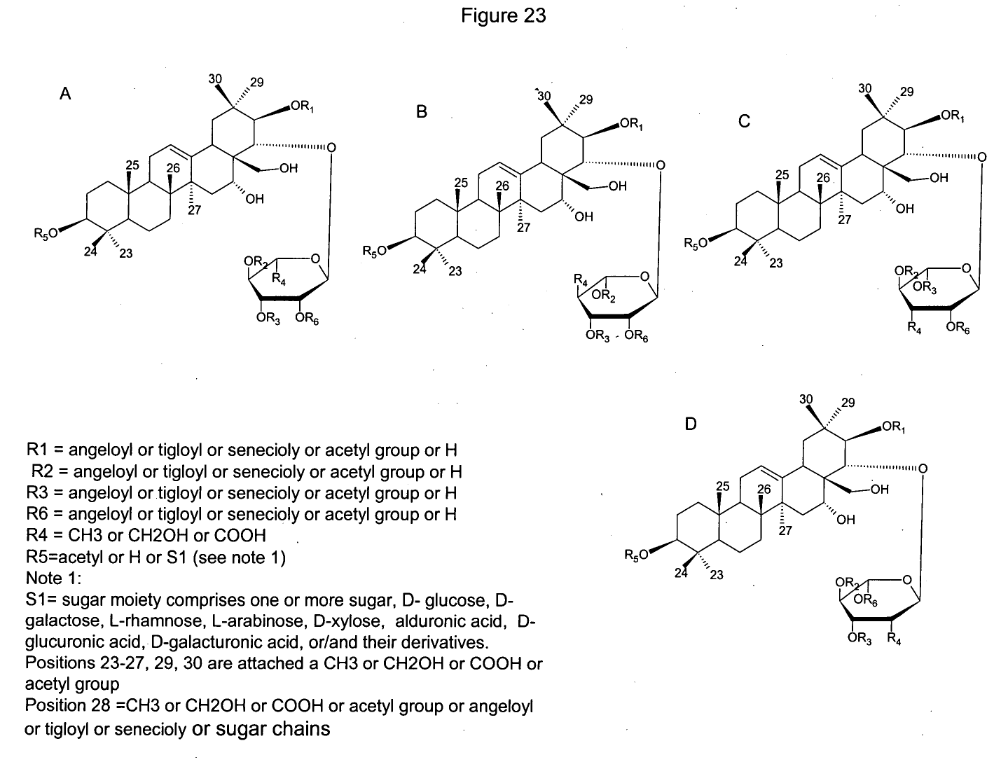

FIG. 23 shows a structure of saponins

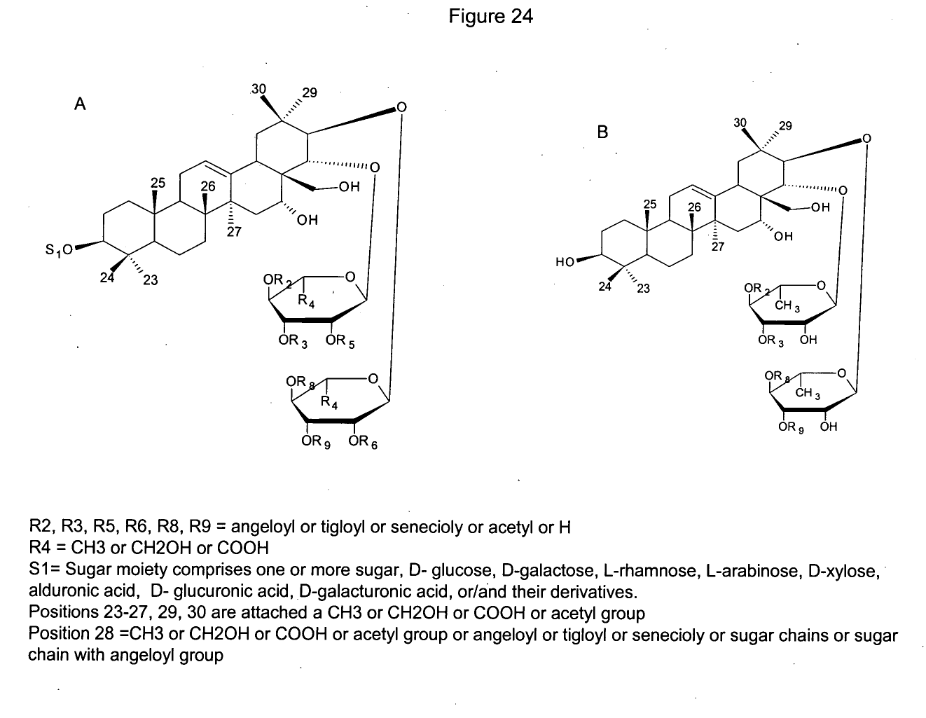

FIG. 24 shows a structure of saponins

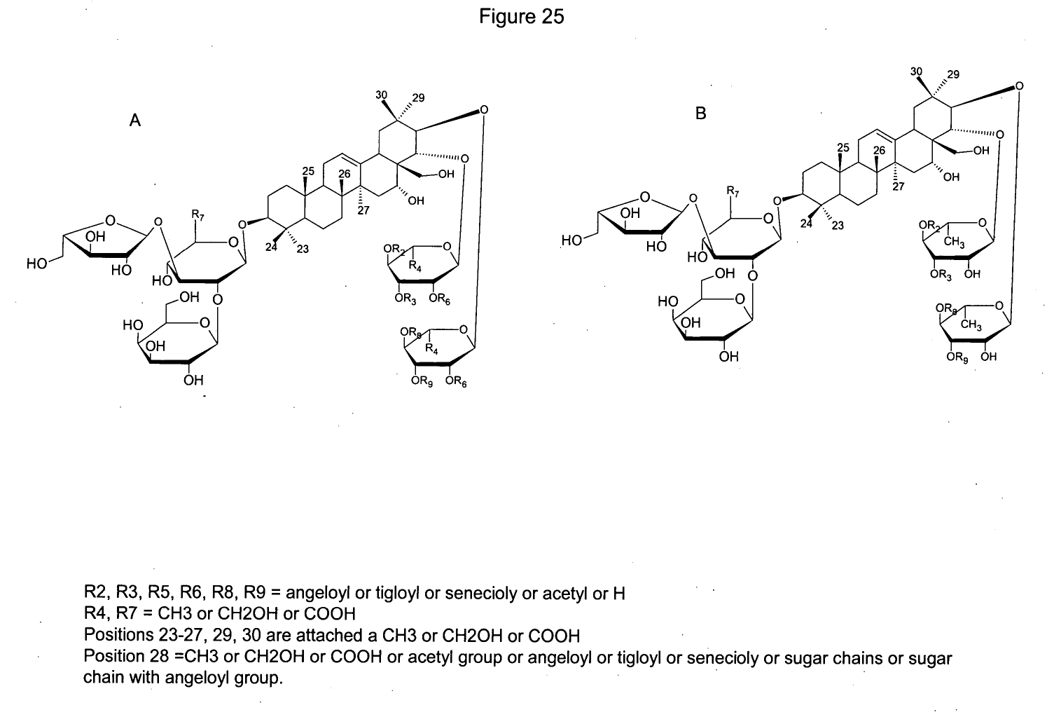

FIG. 25 shows a structure of saponins

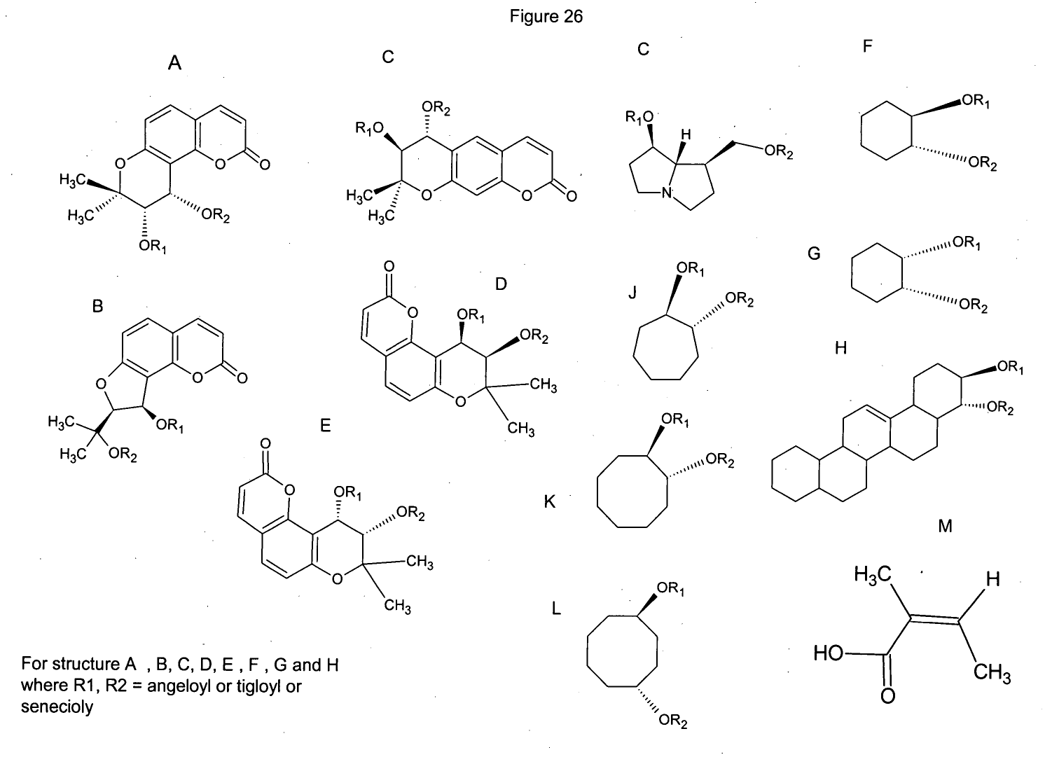

FIG. 26 shows a structure of compounds with angeloyl groups

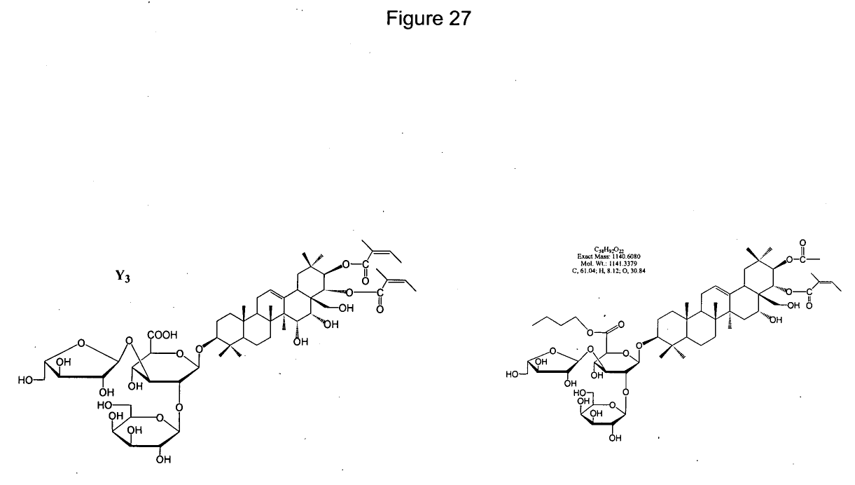

FIG. 27 shows a structure of saponins

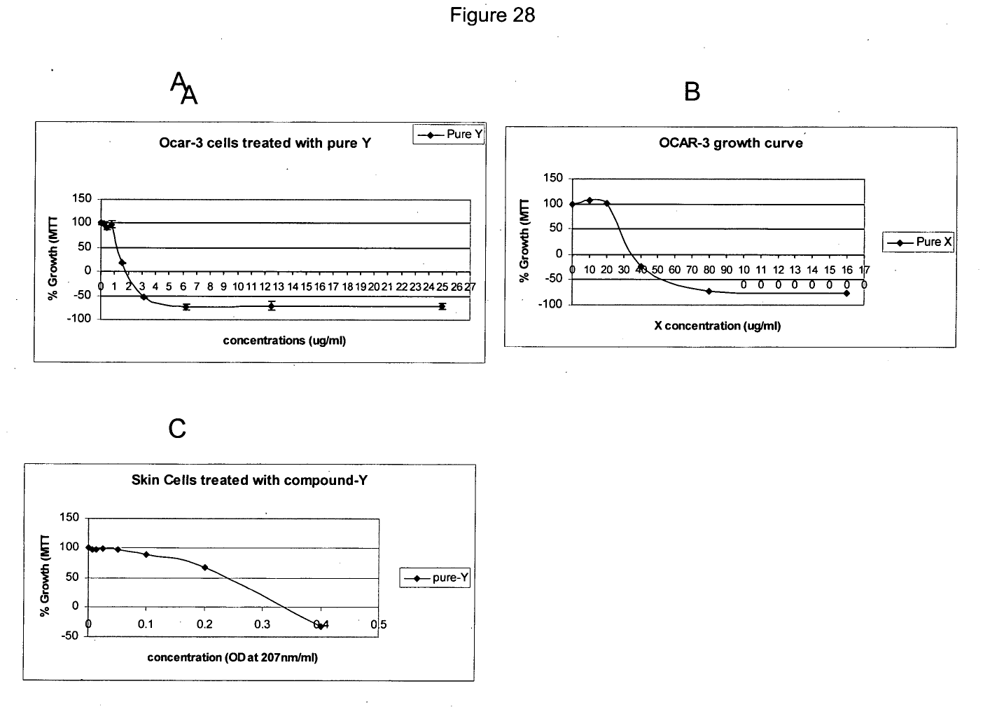

FIG. 28A and B shows the comparison of potency of Compound Y (saponin with 2 angeloyl groups) and compound X (saponin with 1 angeloyl) in ovarian cancer cells. The IC50 for Compound Y in ovary cells is about 1.5 ug/ml while the IC50 for compound X is 30 ug/ml.

FIG. 28C shows the inhibition of the purified Compound Y on the growth of skin cancer cell. The IC50 is 0.23 ug/ml.

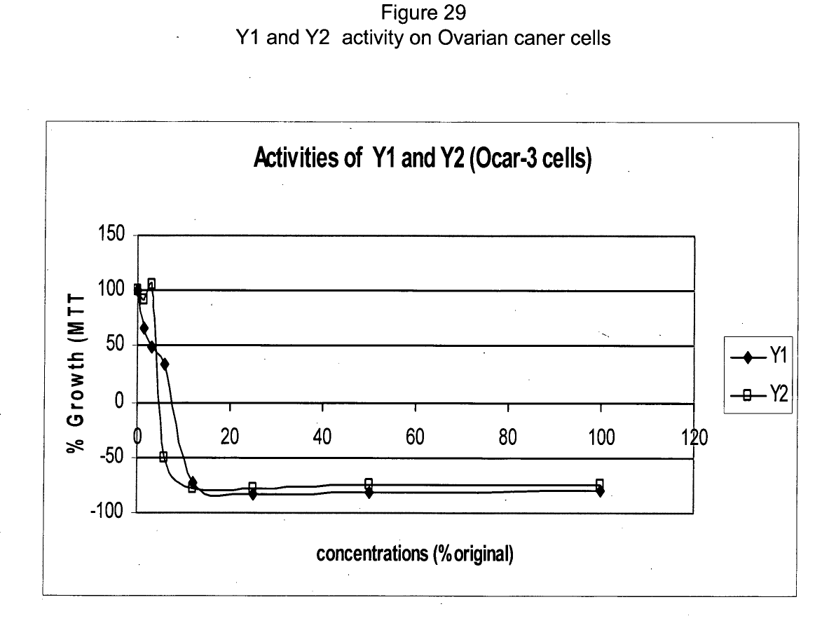

FIG. 29 shows the inhibition of the purified Compound Y1 and Compound Y2 on the growth of ovarian cancer cells.

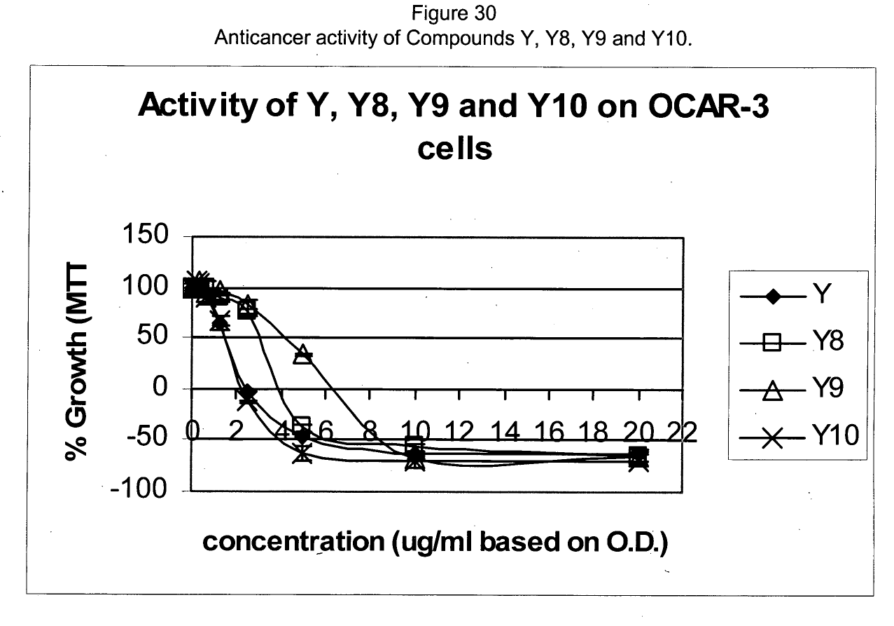

FIG. 30 shows the anticancer activity of Y, Y8, Y9 and Y10 on ovarian cancer cells as determined by MTT assay.

### DETAILED DESCRIPTION OF THE INVENTION

This invention describes the results of a program of screening the bioactive compounds from natural plants. Most of the plants are in Sapindaceae family, which has 1400-2000 species with 140-150 genera. The program of screening for bioactive compounds is based on our purification methods and biological assays including the MTT assay.

The invention provides methods and uses of saponins including triterpenoidal saponins purified or isolated from plants in the following genus:

Acer, Aesculus, Alectryon, Allophylus, Allosanthus, Amesiodendron, Aphania, Aporrhiza, Arfeuillea, Arytera, Atalaya, Athyana, Averrhoidium, Blighia, Boniodendron, Camellia, Camptolepis, Cardiospermum, Castanospora, Chonopetalum, Chouxia, Chytranthus, Conchopetalum, Cossinia, Cubilia, Cupania, Cupaniopsis, Deinbollia, Delavaya, Diatenopteryx, Dictyoneura, Dilodendron, Dimocarpus, Diploglottis, Diplokelepa, Diplopeltis, Dipteronia, Distichostemon, Dodonaea, Doratoxylon, Elattostachys, Eriocoelum, Erioglossum, Erythrophysa, Euchorium, Euphorianthus, Eurycorymbus, Exothea, Filicium, Ganophyllum, Glenniea, Gloeocarpus, Gongrodiscus, Gongrospermum, Guindilia, Guioa, Handeliodendron, Haplocoelum, Harpullia, Hippobromus, Hornea, Houssayanthus, Hypelate, Hypseloderma, Jagera, Koelreuteria, Laccodiscus, Lecaniodiscus, Lepiderema, Lepidopetalum, Lepisanthes, Litchi, Llagunoa, Lophostigma, Loxodiscus, Lychnodiscus, Macphersonia, Maesa, Magonia, Majidea, Matayba, Melicoccus, Mischocarpus, Molinaea, Negundo, Neotina, Nephelium, Otonephelium, Otophora, Pappea, Paranephelium, Paullinia, Pavieasia, Pentascyphus, Phyllotrichum, Pittosporum, Placodiscus, Plagioscyphus, Podonephelium, Pometia, Porocystis, Pseudima, Pseudopancovia, Pseudopteris, Ptelea, Radikofera, Rhysotoechia, Sapindus, Sarcopteryx, Sarcotoechia, Scyphonychium, Serjania, Sisyrolepis, Smelophyllum, Stadmania, Stocksia, Storthocalyx, Synima, Talisia, Thinouia, Thouinia, Thouinidium, Tina, Tinopsis, Toechima, Toulicia, Trigonachras, Tripterodendron, Tristira, Tristiropsis, Tsingya, Ungnadia, Urvillea, Vouarana, Xanthoceras, Xeropspermum, Zanha, Zollingeria.

Saponins including triterpenoidal saponins may also be purified or isolated from the following species of plants:

Acer campestre L., Acer chienii Hu et Cheng, Acer chingii Hu, Acer davidii Franch, Acer laxiflorum Pax, Acer mandshuricum Maxim., Acer mono Maxim., Acer orientale L., Acer palmatum Thunb., Acer sinense Pax, Acer wilsonii Redhd., Acer yui Fang, Aesculus arguta, Aesculus assamica Griff., Aesculus californica (Spach) Nutt., Aesculus chinensis Bunge, Aesculus chinensis var. Chekiangensis (Hu et Fang) Fang, Aesculus chuniana Hu et Fang, Aesculus flava (A. octandra), Aesculus glabra Willd., Aesculus hippocastanum, Aesculus indica, Aesculus lantsangensis Hu et Fang, wangii Aesculus megaphylla Hu et Fang, chinensis Aesculus neglecta, Aesculus octandra Marsh., Aesculus parviflora, Aesculus pavia, Aesculus polyneura Hu et Fang, Aesculus tsiangui Hu et Fang, Aesculus sylvatica, Aesculus turbinata, Aesculus wangii Hu, Aesculus wangii var. ruticola Hu et Fang, Aesculus wilsonii, Allophylus caudatus Radlk. [A. racemosus auct. Non(L.) Radlk], Allophylus chartaceus (Kurz.)Radik., Allophylus cobbe (Linn.) Raeuch. var. velutinus Corner, Allophylus dimorphus Radik., Allophylus hirsutus Radik., Allophylus longipes Radik., Allophylus petelotii Merr., Allophylus repandifolius Merr. et Chun, Allophylus timornsis (DC.) BI., Allophylus tricophyllus Merr. et Chun, Allophylus viridis Radlk., Amesiodendron chinense (Merr.) Hu, Amesiodendron integrifoliolatum H. S. Lo, Amesiodendron tienlinense H. S. Lo, Aphania oligophylla (Merr. et Chun) H. S. Lo, Aphania rubra (Roxb.) Radlk., Arytera littoralis BI., Blighia sapida, Boniodendron minus (Hemsl.) T. Chen, Camellia axillaris Roxb. ex Ker, Camellia cordifolia (Mech.) Hakai, Camellia edithae Hance, Camellia irrawadiensis Barua, Camellia pitardii Coh. Stuart, Camellia reticulata Lindl., Camellia rosthorniana Hand.-Mazz., Camellia sinensis O. Ktze., Camellia tenii Sealy, Camellia tsaii Hu, Camellia wardii Kobuski, Camellia yunnanensis Coh. Stuart, Cardiospermum halicacabum L., Cupaniopsis anacardioides, Delavaya toxocarpa Franch., Dimocarpus confinis (How et Ho) H. S. Lo, Dimocarpus fumatus (BI.) Leenh. subsp. cacicola C. Y. Wu, Dimocarpus longan Lour.(Euphoria ongan Lour.) Steud., Dimocarpus yunanensis (W. T. Wang) C. Y. Wu et T. Y. Ming, Dipteronia dyerana Henry, Dipteronia sinensis Oliv., Dipteronia sinensis Oliv. var. taipeiensis Fang et Fang f., Dodonaea microzyga, Dodonaea viscosa (L) Jacq.[Ptelea viscosa L.], Erioglossum rubiginosum (Roxb.) BI., Erythrophysa alata, Eurycorymbus austrosinensis Hand.-Mazz., Eurycorymbus cavaleriei (Lével.) Rehd. et Hand.-Mazz., Handeliodendron bodnieri (LévI.) Rehd., Harpullia alata F. Mueller, Harpullia arborea (Blanco) Rdik., Harpullia austro-calcdonica Baillon, Harpullia camptoneura Radlk., Harpullia cauliflora K. Schum. & Lauterb., Harpullia crustacea Radik., Harpullia cupanoides Roxb., Harpullia frutescens F. M. Bailey, Harpullia giganteacapsula M. Vente, Harpullia hillii F. Muell., Harpullia hirsuta Radlk., Harpullia largifolia Radlk., Harpullia leptococca Radlk., Harpullia myrmecophila Merr. & Perry, Harpullia longipetala Leench, Harpullia peekeliana Melch., Harpullia pendula Planch. ex F. muell., Harpullia petiolaris Radlk., Harpullia ramiflora Radlk., Harpullia rhachiptera Rdik., Harpullia rhyticarpa C. T. White & Francis, Harpullia solomenensis M. Vente, Harpullia vaga Merr. & Perry, Hypelate trifoliate, Koelreuteria apiculata Rehd. et Wils., Koelreuteria bipinnata Franch., Koelreuteria bipinnata var. integrifoliola (Merr.) T. chen (K. integrifoliola Merr.), Koelreuteria elegans (Seem.) A. C. Smith susp. formosana (Hayata) Meye, Koelreuteria monor Hemsl., Koelreuteria paniculata Laxm., Lepisanthes basicardia Radk., Lepisanthes browniana Hiern, Lepisanthes hainanensis H. S. Lo, Litchi chinensis Sonn., Maesa hupehensis Rehl., Maesa japonica (Thunb.) Moritzi, Maesa lanceolata, Maesa laxiflora, Maesa montana A. DC., Maesa perlarius (Lour.) Merr. Maesa tenera Mez, Melicoccus bijuatus, Mischocarpus hainanensis H. S. Lo, Mischocarpus pentapetalus (Roxb.) Radlk., Mischocarpus sundaicus BI., Nephelium Chryseum BI., Nephelium lappaceum, Nephelium topengii (Merr.) H. S. Lo, Otophora unilocularis (Leenh.) H. S. Lo, Paranephelium hainanensis H. S. Lo, Paranephelium hystrix W. W. Smith, Pavieasia kwangsiensis H. S. Lo, Pavieasia yunnanensis H. S. Lo, Pittosporum balancae DC., Pittosporum brevicalyx (Oliv.) Gagnep., Pittosporum crassifolium A. Cunn., Pittosporum crispulum Gagnep., Pittosporum daphyniphylloides Hayata, Pittosporum elevaticostatum H. T. Chang et Yan, Pittosporum eugennioides A. Cunn., Pittosporum glabratum Lindl., Pittosporum glabratum Lindl. var. neriifolium Rehd., Pittosporum heterophyllum Franch., Pittosporum illicioides Makino, Pittosporum kerrii Craib, Pittosporum kunmingense H. T. Chang et Yan, Pittosporum leptosepalum Gowda, Pittosporum napaulense (DC.) Rehd. et Wils., Pittosporum omeiense H. T. Chang et Yan, Pittosporum ovoideum Gowda, Pittosporum parvicapsulare H. T. Chang et Yan, Pittosporum pauciflorum Hook. et Arn., Pittosporum pentandrum var. hainanense (Gangnep.) H. L. Li, Pittosporum perryanum Gowda, Pittosporum phillyraeoides DC., Pittosporum planilobum H. T. Chang et Yan, Pittosporum podocarpum Gagnep., Pittosporum podocarpum Gagnep., Pittosporum pulchrum Gagnep., Pittosporum rehderianum Gowda, Pittosporum rhombifolium A. Cunn. ex Hook., Pittosporum sahnianum Gowda, Pittosporum subulisepalum Hu et Wang, Pittosporum tenuifolium Gaertn., Pittosporum tobira (Thunb.) Ait., Pittosporum tobira (Thunb.) Ait., var. calvescens Ohwi, Pittosporum tonkenese Gagnep., Pittosporum trigonocarpum LévI., Pittosporum truncatum Pritz., Pittosporum undulatifolium H. T. Chan et Yan, Pittosporum undulatum Venten., Pittosporum viridiflorum, Pittosporum xylocarpum Hu et Wang, Pometia pinnata J. R. et G. Forst., Ptelea trifoliate, Ptelea viscosa Linn., Sapindus abruptus Lour., Sapindus Chinesis Murray, Sapindus delavayi (Franch.) Radlk. [Pancovia delavayi Franch], Sapindus mukorossi Gaertn., Sapindus rarak DC., Sapindus rarak DC., var. velutinus C. Y. Wu, Sapindus saponaria var. drummondii, Sapindus tomentosus Kurz, Ungnadia speciosa, Xanthoceras sorbifolia Bunge. Xeropspermum bonii (Lecomte) Radik.

This invention provides a compound comprising the structures recited in FIGS. 1 to 27. This invention provides a compound comprising a triterpenoidal saponin, triterpenoid, triterpenoidal compound or sapongenin, comprising two angeloyl group or at least two side groups selected from the group consisting of: angeloyl groups, tigloyl groups and senecioyl groups, wherein the side groups are attached to carbon 21 and 22 of triterpenoidal saponin, triterpenoid, triterpenoidal compound or sapongenin backbone. In an embodiment, the saponin comprising a sugar moiety, wherein the sugar moiety comprises at least one sugar, or D-glucose, or D-galactose, or L-rhamnose, or L-arabinose, or D-xylose, or alduronic acid, or D-glucuronic acid or D-galacturonic acid, or their derivative thereof, or the combination thereof. A sugar moiety is a segment of molecule comprising one or more sugar group. The above compounds are obtainable from the above-described plants. The compounds comprising the structure in FIG. 1 to 27 are obtainable from the above-described plants.

This invention further provides composition comprising the structures recited in FIGS. 1 to 27, or a compound comprising a triterpenoidal saponin, triterpenoid, triterpenoidal compound or sapongenin, comprising at least two side groups selected from the group consisting of: angeloyl groups, tigloyl groups and senecioyl groups, wherein the side groups are attached to carbon 21, 22 or 28 of triterpenoidal saponin, triterpenoid, triterpenoidal compound or sapongenin backbone. These compositions are obtainable from the above-identified plants.

This invention further provides composition comprising the structures recited in FIGS. 1 to 27, or a compound comprising two angeloyl groups or at least two side groups selected from the group consisting of: angeloyl groups, tigloyl groups and senecioyl groups,

This invention provides uses of the saponins isolated from the roots, kernel, leave, bark, stem, husk, seed, seed shell or fruit of the above plants, and methods of their preparations.

This invention provides a method of preparing the saponins, comprising the steps of:

(a) extracting roots, kernel, leave, bark, stem, husk, seed, seed shell or fruit or combinations thereof of the above plant with organic solvents such as ethanol or methanol to obtain a organic extract;

(b) collecting the organic extract;

(c) refluxing the organic extract to obtain a second extract;

(d) removing the organic solvent from the second extract to obtain a third extract;

(e) drying and sterilizing the third extract to obtain a crude extract powder;

(f) fractionating the crude extract powder into fractions or components. Fractionation may be achieved by HPLC and FPLC chromatography with silica gel, C18 or other equivalent solid phase materials;

(g) monitoring the fractions. If using HPLC or FPLC, absorption wavelength at 207 nm to 500 nm may be used;

(h) identifying the bioactive components of the crude extract;

(i) purifying one or more bioactive components of the crude extract with chromatographic techniques that employ FPLC to obtain one or more fraction of the bioactive component; and

(j) isolating the bioactive components with chromatographic techniques that employ preparative columns and HPLC.

The following is an example of methods and materials that were used to test the bioactivities of Saponins or compounds of this invention.

Cells. Human cancer cell lines were obtained from American Type Culture Collection: HTB-9 (bladder), HeLa-S3 (cervix), DU145 (prostate), H460 (lung), MCF-7 (breast), K562 (leukocytes), HCT116 (colon), HepG2 (liver), U2OS (bone), T98G (brain) and OVCAR-3 (ovary). Cells were grown in culture medium (HeLa-S3, DU145, MCF-7, Hep-G2 and T98G in MEN (Earle's salts); HTB-9, H460, K562, OVCAR-3 in RPMI-1640; HCT-116, U2OS in McCoy-5A) supplemented with 10% fetal calf serum, glutamine and antibiotics in a 5% CO₂ humidified incubator at 37° C.

MTT Assay. The procedure for MTT assay followed the method described in (Carmichael et al., 1987) with modifications. Cells were seeded into a 96-wells plate at concentrations of 10,000/well (HTB-9, HeLa, H460, HCT116, T98G, OVCAR-3), 15,000/well (DU145, MCF-7, HepG2, U2OS), or 40,000/well (K562), for 24 hours before drug-treatment. Cells were then exposed to drugs for 48 hours (72 hours for HepG2, U2OS, and 96 hours for MCF-7). After the drug-treatment, MTT (0.5 mg/ml) was added to cultures for an hour. The formation of formazan (product of the reduction of tetrazolium by viable cells) was dissolved with DMSO and the O. D. at 490 nm was measured by an ELISA reader. The MTT level of cells before drug-treatment was also measured (T0). The % cell-growth (% G) is calculated as:
% G=(TD−T0/TC−T0)×100 (1),
where TC or TD represent O. D. readings of control or drug-treated cells. When T0>TD, then the cytotoxicity (LC) expressed as % of the control is calculated as:
% LC=(TD−T0/T0)×100 (2)

This invention provides a composition effective in reducing or inhibiting cancer growth. The cancer includes but is not limited to bladder cancer, bone cancer and ovary cancer.

This invention provides a composition comprising triterpenoidal saponins or their derivatives for inhibiting tumor growth.

This invention provides a compound selected from a compound of formula (1): 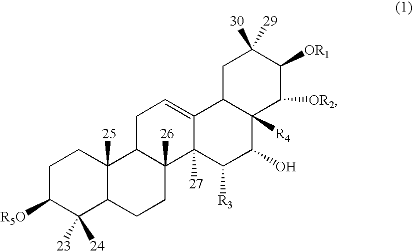
or a salt, ester, metabolite or derivative thereof, wherein R1 and R2 represent angeloyl group; R3 represents H or OH; R4 represent CH2OR6; and wherein R6 is H; R5 represents at least one sugar moiety or its derivatives. In an embodiment, R1 and R2 represent angeloyl group; R3 represents H or OH; R4 represents COOR6 wherein R6 is H; R5 represents at least one sugar moiety or its derivatives. In an embodiment, R1 represents H; R2 represents angeloyl group; R3 represents H or OH; R4 represents CH2OR6 or COOR6; wherein R6 is an angeloyl group; and R5 represents at least one sugar moiety or its derivatives.

In another embodiment, at least two of R1, R2, and R6 comprise an angeloyl group or acid having five carbons; R3 represents H or OH; R4 represents CH2OR6 or COOR6; and wherein R6 is angeloyl group, H, acetyl group, tigloyl group, senecioly group, or an acid having two to five carbons; R5 represents at least one sugar moiety or its derivatives. In a further embodiment, at least one angeloyl of R1 or R2 is replaced by acetyl group, tigloyl group, senecioly group, or an acid having two to five carbons; R3 represents H or OH; R4 represents CH2OR6 or COOR6; and wherein R6 is angeloyl group; R5 represents at least one sugar moiety or its derivatives. In a further embodiment, at least one of R1, R2, and R6 is a sugar moiety or rhamnose comprising at least two angeloyl groups, acetyl group, tigloyl group, senecioly group, or an acid having two to five carbons or combination thereof. In a further embodiment, positions 23, 24, 25, 26, 29, 30 of the compound independently comprise CH3, CH2OH, CHO, COOH, alkyls group, acetyl group or derivative thereof. In a further embodiment, R5 represents sugar moiety comprising glucose, galactose or arabinose. In a further embodiment, R5 represents sugar moiety, wherein the sugar moiety comprises at least one sugar, or D-glucose, D-galactose, or L-rhamnose, or L-arabinose, or D-xylose, or alduronic acid, or D-glucuronic acid or D-galacturonic acid, or derivative thereof, or the combination thereof. In an embodiment, R5 represents a compound capable of performing the function of the sugar moiety. In a further embodiment, the R5 represents H. In a further embodiment, R4 represents H or OH or CH3. In a further embodiment, R1 or/and R2 is a functional group capable of performing the function of the angeloyl. R5 represents a compound capable of performing the function of the sugar moiety.

A sugar moiety is a segment of molecule comprising one or more sugar group. Substitution, deletion and/or addition of any group in the above-described compounds will be apparent to one of ordinary skill in the art based on the teaching of this application. In a further embodiment, the substitution, deletion and/or addition of the group(s) in the compound of the invention does not substantially affect the biological function of the compound. In a further embodiment, the angeloyl groups are in a trans-position on a structure.

This invention provides a compound selected from a compound of formula (2): 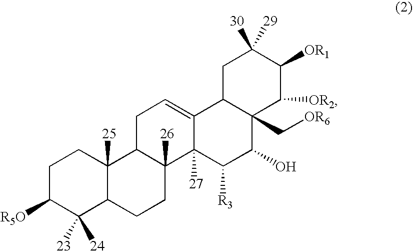
or a salt, ester or derivative thereof, wherein R1 represents angeloyl group; R2 represents angeloyl group; R3 represents OH or H; Positions 23, 24, 25, 26, 27, 29, 30 of the compound independently comprise CH3, or CH2OH, or CHO, or COOH, alkyls group, or acetyl group, or derivative; R6 represents Ac or H; and R5 represents sugar moiety, wherein the sugar moiety comprises at least one sugar, or D-glucose, or D-galactose, or L-rhamnose, or L-arabinose, or D-xylose, or alduronic acid, or D-glucuronic acid, or D-galacturonic acid, or their derivative thereof, or the combination thereof. In an embodiment, R5 represents a compound capable of performing the function of the sugar moiety. In another embodiment the sugar moiety comprises L-arabinose, D-glucose and/or D-galactose, or combinations thereof. In a further embodiment, any two of R1, R2 or R6 are angeloyl groups, or any one of R1, R2 or R6 is attached to a sugar moiety in which two angeloyl groups are attached to adjacent carbons of the monosaccharides. In a further embodiment, R1, R2, and R6 comprises angeloyl group, acetyl group, tigloyl group, senecioly group, or an acid with two to five carbons or combibation thereof. In a further embodiment, at least one of R1, R2 or R6 is attached a sugar moiety or rhamnose, wherein sugar moiety or rhamnose comprises two angeloyl group, acetyl group, tigloyl group, senecioly group, acid having two to five carbons, or combinations thereof.

This invention provides a compound selected from a compound of formula (3): 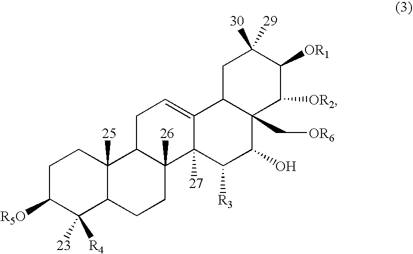
or a salt, ester or derivative thereof, wherein R1 represents angeloyl group; R2 represents angeloyl group; R3 represents OH or H; R4 represents CH3 or CH2OH or alkyls group or their derivatives; R6 represents Ac or H and R5 represents sugar moiety, wherein the sugar moiety comprises at least one sugar, or D-glucose, or D-galactose, or L-rhamnose, or L-arabinose, or D-xylose, or alduronic acid, or D-glucuronic acid, or D-galacturonic acid, or derivative thereof, or the combination thereof. In an embodiment, R5 represents a compound capable of performing the function of sugar moiety. In another embodiment the sugar moiety comprises L-arabinose, or D-glucose, or D-galactose, or combinations thereof. In a further embodiment, at least one of R1, R2 or R6 is attached a sugar moiety or rhamnose, wherein sugar moiety or rhamnose comprises two angeloyl group, acetyl group, tigloyl group, senecioly group, acid having two to five carbons, or combinations thereof.

This invention provides a compound selected from a compound of formula (4): 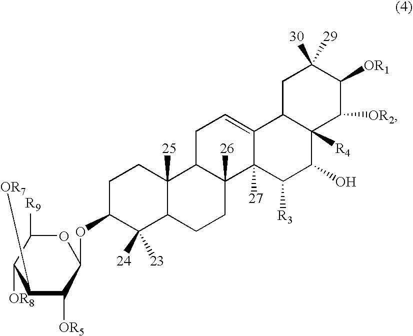
or a salt, ester, metabolite or derivative thereof, wherein R1 and R2 represent angeloyl group; R3, represents H or OH; R4 represent CH2OR6; and wherein R6 is H or acetyl; R5 represents sugar moiety or D-glucose; R7 represents a sugar moiety or L-arabinose; R8 represents sugar moiety or D-galactose; R9 represent COOH or CH2OH.

In an embodiment, R4 represents COOR6, wherein the R6 is H or acetyl. In an embodiment, the R5, R7 or/and R8 are H or sugar moiety, wherein the sugar moiety comprises at least one sugar, or glucose, or galactose, or rhamnose, or arabinose, or xylose, or alduronic acid, or glucuronic acid or galacturonic acid, or derivative thereof. In an embodiment, at least 2 of R1, R2 and R6 are angeloyl group; R4 represent CH2OR6 or COOR6, wherein R6, R1 and R2 are angeloyl group, acetyl group, tigloyl group, senecioly group, an acid having two to five carbons or H. In a further embodiment, at least two of R1, R2 and R6 are angeloyl group, acetyl group, tigloyl group, senecioly group, or an acid having two to five carbons; R3 represents H or OH; R4 represents CH2OR6 or COOR6, where R6 is H or angeloyl group, acetyl group, tigloyl group, senecioly group.

In an embodiment, R1 and R2 represent angeloyl group; R3 represents H or OH; R4 represents COOR6 wherein R6 is H;

In an embodiment, R1 represents H; R2 represents angeloyl group; R3 represents H or OH; R4 represents CH2OR6 or COOR6; wherein R6 is an angeloyl group.

In another embodiment, at least two of R1, R2, and R6 comprise an angeloyl group; R3 represents H or OH; R4 represents CH2OR6 or COOR6; and wherein R6 is H, angeloyl group, acetyl group, tigloyl group, senecioly group, or an acid having two to five carbons.

In a further embodiment, at least one angeloyl of R1 or R2 is replaced by acetyl group, tigloyl group, senecioly group, or an acid having two to five carbons; R3 represents H or OH; R4 represents CH2OR6 or COOR6; and wherein R6 is angeloyl group.

In a further embodiment, at least one of R1, R2, and R6 is a sugar moiety or rhamnose comprising at least two angeloyl groups, acetyl group, tigloyl group, senecioly group, or an acid having two to five carbons or combination thereof. In a further embodiment, positions 23, 24, 25, 26, 29, 30 of the compound independently comprise CH3, CH2OH, CHO, COOH, alkyls group, acetyl group or derivative thereof. In a further embodiment, R4 represents H or OH or CH3. A sugar moiety is a segment of molecule comprising one or more sugar group.

This invention provides a compound selected from a compound of formula (5): 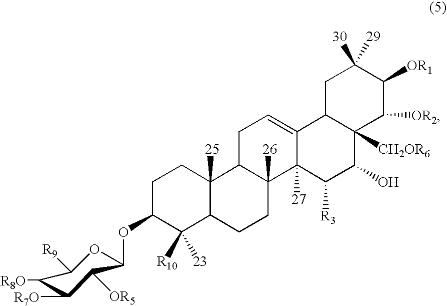
or a salt, ester, metabolite or derivative thereof, wherein R1 and R2 represent angeloyl group; R3 represents H or OH; R6 represent H or acetyl; R9 represents COOH or CH2OH; R10 represent CH3 or CH2OH or COOH; R5, R7 and R8 are H or/and sugar moiety, wherein the sugar moiety comprises at least one sugar, D-glucose, D-galactose, L-rhamnose, L-arabinose, D-xylose, alduronic acid, D-glucuronic acid, or D-galacturonic acid, or derivative thereof. In an embodiment, at least one of R1, R2, and R6 is a sugar moiety or rhamnose comprising at least two angeloyl groups, acetyl group, tigloyl group, senecioly group, or an acid having two to five carbons or combination thereof. In another embodiment, at least two of R1, R2, and R6 comprise an angeloyl group

This invention provides a compound selected from a compound of formula (6): 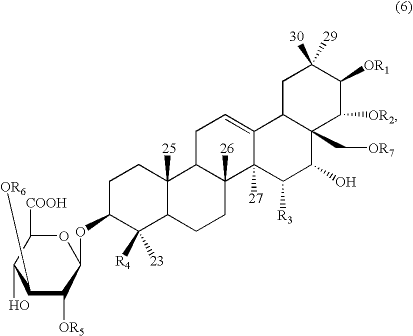
or a salt, ester or derivative thereof, wherein R1 represents angeloyl group; R2 represents angeloyl group; R3 represents OH or H; R4 represents CH3 or CH2OH; R7 represents H; and R5 represents D-glucose, D-Galactose, L-arabinose or H; and R6 represents D-glucose, D-Galactose, L-arabinose or H. In anbodiment, R5 or/and R6 are H or sugar moiety comprises at least one sugar, D-glucose, D-galactose, L-rhamnose, L-arabinose, D-xylose, alduronic acid, D-glucuronic acid, or D-galacturonic acid, or derivative thereof.

A sugar moiety is a segment of molecule comprising one or more sugar group. Substitution, deletion and/or addition of any group in the above-described compounds will be apparent to one of ordinary skill in the art based on the teaching of this application. In a further embodiment, the substitution, deletion and/or addition of the group(s) in the compound of the invention does not substantially affect the biological function of the compound.

This invention provides a method of inhibiting tumor cell growth comprising administering to a subject, in need thereof, an appropriate amount of triterpenoidal saponins comprising two or more angeloyl groups or comprising the structure of FIGS. 1-27.

This invention provides a composition comprising the compounds as described above effective in reducing or inhibiting cancer growth. The cancer includes but is not limited to bladder cancer, bone cancer and ovarian cancer.

The saponins isolated from Xanthoceras sorbifolia with the characteristic structure described in the present invention can be used for anti-cancer therapy. The cancer includes but is not limited to bladder cancer, bone cancer and ovarian cancer.

This invention provides a composition comprising the above described compounds or their derivatives for treating human immunodeficiency virus (HIV), Severe Acute Respiratory Syndrome (SARS), flu disease or inhibits virus activities. The biologically active triterpenoid saponins structures are shown in FIG. 1

Triterpenoid saponins comprises the formula as following:
3-O-{[β-D-galactopyranosyl (1→2)]-[α-L-arabinofuranosyl (1→3)]-β-D-glucuronopyranoside butyl ester}-21-O-acetyl-22-O-angeloyl-3β,16α,21β,22α,28-pentahydroxyolean-12-ene.
3-O-[β-D-galactopyranosyl(1→2)]-α-L-arabinofuranosyl(1→3)-β-D-glucuronopyranosyl-21,22-O-diangeloyl-3β, 15α, 16α, 21β, 22α, 28-hexahydroxyolean-12-ene,
3-O-[β-D-galactopyranosyl(1→2)]-α-L-arabinofuranosyl(1→3)-β-D-glucuronopyranosyl-21-O-(3,4-diangeloyl)-α-L-rhamnophyranosyl-22-O-acetyl-3β, 16α, 21β, 22α, 28-pentahydroxyolean-12-ene,
3-O-[β-D-glucopyranosyl-(1→2)]-α-L-arabinofuranosyl(1→3)-β-D-glucuronopyranosyl-21,22-O-diangeloyl-3β, 15α, 16α, 21β, 22α, 24β, 28-heptahydroxyolean-12-ene,
3-O-[β-glucopyranosyl (1→2)]-α-arabinofuranosyl (1→3)-β-glucuronopyranosyl-21, 22-O-diangeloyl-3β, 16α, 21β, 22α, 24β, 28-hexahydroxyolean-12-ene,
3-O-[β-galactopyranosyl (1→2)]-α-arabinofuranosyl (1→3)-β-glucuronopyranosyl-21-O-(3,4-diangeloyl)-α-rhamnopyranosyl-28-O-acetyl-3β, 16α, 21β, 22α, 28-pentahydroxyolean-12-ene,
3-O-[β-galactopyranosyl (1→2)]-α-arabinofuranosyl (1→3)-β-glucuronopyranosyl-21, 22-O-diangeloyl-3β, 16α, 21β, 22α, 28-pentahydroxyolean-12-ene,

This invention provides a composition comprising the compounds as described above effective in reducing or inhibiting cancer growth. The cancer includes but is not limited to bladder cancer, bone cancer and ovarian cancer.

The saponins isolated from Harpullia austro-calcdonica with the characteristic structure described in the present invention can be used for anti-cancer therapy. The cancer includes but is not limited to bladder cancer, bone cancer and ovarian cancer.

This invention provides a composition comprising the above described compounds or their derivatives for inhibiting human immunodeficiency virus (HIV), Severe Acute Respiratory Syndrome (SARS), flu disease or inhibits virus activities.

The biologically active triterpenoid saponins structures are shown in FIG. 11A. See also Phytochemistry 59 (2002) 825-832, Triterpenoid saponins and acylated prosapogenins from Harpullia austro-calcdonica.

Wherein R1=R2=angeloyl group; R3=CH2OH or CH3 or CHO.

The biologically active triterpenoid saponins structures are shown in FIG. 15. See also Phytochemistry 66 (2005) 825-835, Haemolytic acylated triterpenoid saponins from Harpullia austro-calcdonica.

Triterpenoid saponins isolated from seeds of Aesculus chinensis having the characteristic structure(s) as disclosed in the present invention can be used in anti-cancer therapy. The cancer that triterpenoid saponins is effective against includes but is not limited to bladder cancer, bone cancer and ovarian cancer. This invention provides a composition comprising the above-described compounds and their derivatives for inhibiting cancer, Severe Acute Respiratory Syndrome (SARS), flu disease or inhibits virus activities.

Triterpenoidal saponins comprise the structures shown or described in FIG. 7A.

| Wherein | R1 | R2 | R3 |
|---|---|---|---|
| 1 | Tigloyl | Acetyl | H |
| 2 | Angeloyl | Acetyl | H |
| 3 | Tigloyl | H | H |
| 4 | Angeloyl | H | Acetyl |
| 5 | H | Tigloyl | Acetyl |
| 6 | H | Angeloyl | Acetyl |
| 7 | H | H | Tigloyl |
| 8 | H | H | Angeloyl |

See also J. Nat. Prod. 1999, 62, 1510-1513.Anti-HIV-1 Protease Triterpenoid Saponins from the seed of Aesculus chinensis.

Triterpenoid saponins isolated from Aesculus, Aesculus arguta, Aesculus assamica Griff., Aesculus californica (Spach) Nutt., Aesculus chinensis Bunge, Aesculus chinensis var. Chekiangensis (Hu et Fang) Fang, Aesculus chuniana Hu et Fang, Aesculus flava (A. octandra), Aesculus glabra Wilid., Aesculus hippocastanum, Aesculus indica, Aesculus lantsangensis Hu et Fang, wangii Aesculus megaphylla Hu et Fang, chinensis Aesculus neglecta, Aesculus octandra Marsh., Aesculus parviflora, Aesculus pavia, Aesculus polyneura Hu et Fang, Aesculus tsianguii Hu et Fang, Aesculus sylvatica, Aesculus turbinata, Aesculus wangii Hu, Aesculus wangii var. ruticola Hu et Fang or Aesculus wilsonii, having the characteristic structure(s) as disclosed in the present invention can be used in anti-cancer therapy. The cancer that triterpenoid saponin is effective against includes but is not limited to bladder cancer, bone cancer and ovarian cancer. This invention provides a composition comprising the above-described compounds and their derivatives for inhibiting cancer, Severe Acute Respiratory Syndrome (SARS), flu disease or inhibits virus activities.

Triterpenoid saponins comprise the structures shown or described in FIG. 7A, 7B, 7C, 7D.

Wherein R1=angeloyl group or tigloyl group or senecioyl group or acetyl or H.

R2=angeloyl group or tigloyl group or senecioyl group or acetyl group or acetyl or H.

R3=angeloyl group or tigloyl group or senecioyl group or acetyl group or acetyl or H.

R6=H or OH

Position 23-27 and 28-30 are attached with CH3 or CH2OH or COOH or CHO

Triterpenoid saponins isolated from Aesculus, Aesculus arguta, Aesculus assamica Griff., Aesculus californica (Spach) Nutt., Aesculus chinensis Bunge, Aesculus chinensis var. Chekiangensis (Hu et Fang) Fang, Aesculus chuniana Hu et Fang, Aesculus flava (A. octandra), Aesculus glabra Wild., Aesculus hippocastanum, Aesculus indica, Aesculus lantsangensis Hu et Fang, wangii Aesculus megaphylla Hu et Fang, chinensis Aesculus neglecta, Aesculus octandra Marsh., Aesculus parviflora, Aesculus pavia, Aesculus polyneura Hu et Fang, Aesculus tsianguii Hu et Fang, Aesculus sylvatica, Aesculus turbinata, Aesculus wangii Hu, Aesculus wangii var. ruticola Hu et Fang or Aesculus wilsonii, having the characteristic structure(s) as disclosed in the present invention can be used in anti-cancer therapy. The cancer that triterpenoid saponin is effective against includes but is not limited to bladder cancer, bone cancer and ovarian cancer. This invention provides a composition comprising the above-described compounds and their derivatives for inhibiting cancer, Severe Acute Respiratory Syndrome (SARS), flu disease or inhibits virus activities.

Triterpenoidal saponins comprises the structures shown or described in FIG. 16A, 16B.

Wherein R1=angeloyl or Tigloyl or Senecioyl or acetyl or H

R2=angeloyl or Tigloyl or Senecioyl or acetyl or H

R6=angeloyl or Tigloyl or Senecioyl or acetyl or H

R3=H or OH

R10=CH3 or CH2OH or CHO

R5=sugar moiety or D-glucose or D-galactose or L-rhamnose or L-arabinose or, D-xylose or alduronic acid or D-glucuronic acid or D-galacturonic acid or H

R7=sugar moiety or D-glucose or D-galactose or L-rhamnose or L-arabinose or, D-xylose or alduronic acid or D-glucuronic acid or D-galacturonic acid or H

R8=sugar moiety or D-glucose or D-galactose or L-rhamnose or L-arabinose or, D-xylose or alduronic acid or D-glucuronic acid or D-galacturonic acid or H

R9=COOH or CH2OH

Triterpenoid saponins isolated from the plants described in this invention with the characteristic structure mentioned in this invention can be used to reduce or inhibit cancer growth. The cancer includes but is not limited to bladder cancer, bone cancer and ovarian cancer. This invention also provides a composition comprising the above described compounds or their derivatives capable of inhibiting human immunodeficiency virus (HIV), Severe Acute Respiratory Syndrome (SARS) or flu disease, or capable of inhibiting viral activities.

See structure of compounds in FIG. 17.

Triterpenoid saponins isolated from roots of Camellia sinensis var.assamica, showed in FIG. 13 with the characteristic structure mentioned in this invention is effective in inhibiting or reducing cancer growth. The cancer includes but is not limited to bladder cancer, bone cancer and ovarian cancer.

This invention provides a composition comprising the above described compounds and their derivatives for inhibiting human immunodeficiency virus (HIV), Severe Acute Respiratory Syndrome (SARS), flu disease or inhibits virus activities. See also Phytochemistry 53(2000) 941-946 Triterpenoid saponins from the roots of tea plant (Camellia sinensis var. assamica).

Triterpenoid saponins isolated from Pittosporum viridiflorum with the characteristic structure mentioned in this invention can be used to reduce or inhibit cancer growth. The cancer includes but is not limited to bladder cancer, bone cancer and ovarian cancer. This invention also provides a composition comprising the above described compounds or their derivatives capable of inhibiting human immunodeficiency virus (HIV), Severe Acute Respiratory Syndrome (SARS) or flu disease, or capable of inhibiting viral activities.

See structure of compounds in FIG. 9A and 9B.

Wherein R1=angeloyl group.

R2=senecioyl group.

See also 3:J. Nat. Prod. 2002, 65, 65-68. A New Triterpene Saponin from Pittosporum viridiflorum from Madagascar Rainforest.

The triterpenoid saponins isolated from Pittosporum tobira with the characteristic structure mentioned in this invention can be used for anti-cancer therapy. The cancer includes but is not limited to bladder cancer, bone cancer and ovary cancer. This invention also provides a composition comprising the above described compounds and their derivatives capable of inhibiting human immunodeficiency virus (HIV), Severe Acute Respiratory Syndrome (SARS) or flu disease, or capable of inhibiting viral activities.

See structure of compounds in FIG. 10.

Wherein

| | R1 | R2 | R3 | R4 |
|---|---|---|---|---|
| 1. | 2-acetoxy-2- | acetyl | H | COOH |
| | methybutanoyl | | | |
| 2. | Angeloyl | acetyl | H | COOH |
| 3. | Angeloyl | H | acetyl | COOH |
| 4. | Angeloyl | angeloyl | H | COOH |
| 5 | H | H | H | COOH |
| 7 | H | H | H | COOMe |
| 8 | H | H | H | CH₂OH |

R5=α-L-araf R6=α-L-arap R7=β-D-glup

See also Tetrahedron 58(2002)10127-10136. Isolation and structure elucidation of four new triterpenoid estersaponins from fruit of Pittosporum tobira AIT.

The triterpenoid saponins isolated from Maesa lanceolata with the characterized structure mentioned in this invention can be used to reduce or inhibit cancer growth. The cancer includes but is not limited to bladder cancer, bone cancer and ovarian cancer. This invention also provides a composition comprising the above described compounds or their derivatives capable of inhibiting human immunodeficiency virus (HIV), Severe Acute Respiratory Syndrome (SARS) or flu disease, or capable of inhibiting viral activities.

See structure of compounds in FIG. 14.

Wherein:

| compound | R1 | R2 | R3 |
|---|---|---|---|
| 2 | acetyl | H | angeloyl |
| 3 | H | acetyl | angeloyl |
| 5 | H | propanoyl | angeloyl |
| 7 | H | butanoyl | angeloyl |
| 8 | H | angeloyl | angeloyl |
| 4 | acetyl | acetyl | angeloyl |
| 6 | acetyl | propanoyl | angeloyl |
| 9 | acetyl | butanoyl | angeloyl |
| 10 | acetyl | angeloyl | angeloyl |

See also Phytochemistry 52(1999)1121-1131. New acylated triterpenoid saponins from Maesa lanceolata.

The triterpenoid saponins isolated from Xanthoceras sobifolia with the characteristic structure mentioned in this invention can be used to reduce or inhibit cancer growth. The cancer includes but is not limited to bladder cancer, bone cancer and ovarian cancer. This invention also provides a composition comprising the above described compounds or their derivatives capable of inhibiting human immunodeficiency virus (HIV), Severe Acute Respiratory Syndrome (SARS) or flu disease, or capable of inhibiting viral activities.

See structures of saponins in FIG. 1 to FIG. 6.

See also PCT/US04/043459 and PCT/US04/043465.

This invention provides a method for inhibiting tumor cell growth, human immunodeficiency virus (HIV), Severe Acute Respiratory Syndrome (SARS) or flu disease, or capable of inhibiting viral activities, comprising contacting an effective amount of the compounds in FIG. 1 to FIG. 27. In an embodiment, the above described compound comprising at least two angeloyl groups at carbon 21, 22 and 28. In an embodiment, the above described compound comprising at least two angeloyl groups, tigloyl groups, senecioyl groups or acetyl group or their combinations.

This invention provides a method for inhibiting tumor cell growth, human immunodeficiency virus (HIV), Severe Acute Respiratory Syndrome (SARS) or flu disease, or capable of inhibiting viral activities, comprising contacting an effective amount of the compounds in the FIGS. 3A and B wherein the compound comprises two angeloyl groups at any two of R1, R2 and R4; or the compounds in FIG. 5A, 5B, 6A and 6B wherein the compound comprises two angeloyl groups at any two of R1, R2, R3 and R4; or the compounds in FIGS. 7A, 7B, 7C and 7D wherein the compound comprises two angeloyl groups at any two of R1, R2 and R3; or the compounds in FIG. 8 wherein the compound comprises two angeloyl groups at any two of R1, R2 and R3; or the compounds in FIGS. 9A, 9B and 10 wherein the compound comprises two angeloyl groups at any two of R1, R2 and R3; or the compounds in FIGS. 11, 12 and 13 wherein the compound comprises angeloyl groups at R1 and R2; or the compounds in FIG. 14 wherein the compound comprises two angeloyl groups at any two of R1, R2 and R3; or the compounds in FIG. 15 wherein the compound comprises two angeloyl groups; or the compounds in FIG. 16 wherein the compound comprises two angeloyl groups at any two of R1, R2 and R6; or the compounds in FIG. 16-25 wherein the compound comprises two angeloyl groups.

This invention provides a method for inhibiting tumor cell growth, human immunodeficiency virus (HIV), Severe Acute Respiratory Syndrome (SARS) or flu disease, or capable of inhibiting viral activities, comprising contacting an effective amount of the above described compounds. In an embodiment, the compound is a triterpenoidal saponin or a sapongenin comprising two angeloyl groups attached to carbon 21 and 22 of its sapogenin. In an embodiment, the sapongenin comprising any two of angeloyl groups, tigloyl groups or senecioyl groups, or their combinations thereof attached to carbon 21 and 22 of its sapogenin. In another embodiment, the compound is a triterpenoidal saponin or a sapongenin comprising any two of angeloyl groups, tigeloyl groups or senecioyl groups, or their combinations thereof attached to a sugar moiety which bonds to carbon 21 or 22.

In a further embodiment, the compound is a triterpenoidal saponin or sapongenin comprising at least any one of angeloyl group, tigloyl group, or senecioyl group, or their combinations thereof attached to carbon 21 and/or 22 of its sapogenin. In a further embodiment, the compound is a triterpenoidal saponin or a sapongenin comprising at least two of angeloyl group or tigloyl group or senecioyl group, or their combinations thereof attached to a sugar moiety which bonds to carbon 21 or 22. In an embodiment, the compound is a triterpenoidal saponin or a sapongenin comprising at least two angeloyl groups attached to carbon 21, 22 or 28 of its sapogenin. In another embodiment, the compound is a triterpenoidal saponin or a sapongenin comprising any two of angeloyl groups, tigeloyl groups or senecioyl groups, or their combinations thereof attached to a sugar moiety which bonds to carbon 21, 22 or 28. In an embodiment, the compound is a triterpenoidal saponin or a sapongenin comprising a sugar moiety comprises at least two angeloyl groups attached to carbon 21, 22 or 28 of its sapogenin.

In a further embodiment, the compound is a five ring triterpene comprising at least two angeloyl group, attached to the side chains at one end of the five rings of its sapogenin and a sugar moiety is attached to the side chains of the ring at the other end of the triterpene. In an embodiment the compound comprising at least any two of angeloyl group, tigloyl group, or senecioyl group, or their combinations thereof.

In a further embodiment, the compound is a sapogenin or triterpene comprising at least two angeloyl group, attached to the side chains of its sapogenin and a sugar moiety is attached to the side chains of the triterpene or sapogenin. In an embodiment the compound comprising at least any two of angeloyl group, tigloyl group, or senecioyl group, or their combinations thereof.

In a further embodiment, the compound comprises at least two angeloyl group, attached to the side chains of a compound and a sugar moiety is attached to a side chain of the compound. In an embodiment the compound comprising at least any two of angeloyl group, tigloyl group, or senecioyl group, or their combinations thereof. In an embodiment, the angeloyl can be replaced by a function group which has the function as angeloyl group.

In a further embodiment, a sugar moiety or chain with one or more sugar such as D-glucose, D-galactose, L-rhamnose, L-arabinose, D-xylose, alduronic acid, D-glucuronic acid or D-galacturonic acid, or their combinations thereof, or their derivatives thereof is attached to carbon 3.

In a further embodiment, the compound is a triterpene or sapongenin comprising at least any one of angeloyl group, tigloyl group or senecioyl group, or their combinations thereof attached to it. In a further embodiment, the compound is a triterpene or sapongenin comprising at least one of angeloyl group, tigloyl group or senecioyl group, or their combinations thereof attached to a sugar moiety which bonds to it.

In a further embodiment, at least one sugar moiety with one or more sugar, D-glucose, D-galactose, L-rhamnose, L-arabinose, D-xylose, or alduronic acid, D-glucuronic acid or D-galacturonic acid, or their combinations thereof, or their derivatives thereof is attached to the triterpene. In a further embodiment, bonds 23-30 are attached with CH3 or CH2OH or COOH or acetyl group.

The activities of a saponin compound for regulating or inhibiting tumor cell growth are based on or attributed to its structure that comprises functional group(s) such as angeloyl group, tigloyl group, senecioyl group or acetyl group, or their combinations thereof.

The Compound Y1 and Compound Y2 which comprise with two angeloyl groups show the inhibition on the growth of ovarian cancer cells. See FIG. 29.

The Compound Y, Y8, Y9 and Y10 which comprise with two angeloyl groups show the inhibition on ovarian cancer cells as determined by MTT assay. See FIG. 30.

The compound with single angeloyl group shows weaker anticancer activity than a compound with two angeloyl groups. See FIG. 28.

The compound with two angeloyl groups is more potency than the one with on angeloyl for inhibiting human immunodeficiency virus (HIV), Severe Acute Respiratory Syndrome (SARS), flu disease or inhibits virus activities.

This invention provides a composition comprising the compounds of the invention for treating enuresis and frequency micturition, and for improving the functions of the central nervous system including signaling the bladder to wake up from deep sleep or to relax the bladder so that it can store more urine. The compounds of the invention can be used to relax the detrusor tension caused by aging, stress, nervousness, over-activity, instability, hyper-reflexia, and uninhibited bladder. In another embodiment, the compounds may be used for relaxing the contracted bladder tissue induced by acetylcholine (Ach). The compounds identified and isolated from extract of this invention may be used as acetylcolinesterase, an AChE inhibitor, for regulating Antidiuretic hormone (ADH), which reduces the volume of urine, and as an anti-inflammatory agent.

The compounds of the invention can be used for accelerating the growth of bladder, for suppressing deep sleep, for increasing alterness in a sleeping subject, for modulating the release, breakdown and uptake of Antidieuretic hormone (ADH) and its receptors, for modulating the secretion, breakdown and uptake of Adrenocorticotropic hormone (ACTH) and its receptors, for modulating the release, breakdown and uptake of 5-hydroxytryptamine and its receptors, for modulating the release, breakdown and uptake of Acetycholine (Ach) and its receptors, for modulating the release, breakdown and uptake of Adrenaline (AD) and its receptors, for modulating the release, breakdown and uptake of Dopamine (DA) and its receptors, for modulating the release, breakdown and uptake of Norepinephrine (NE) and its receptors, for preventing sleep paralysis, for modulating the formation, release, breakdown and activity of neuropeptides and their receptors.

This invention provides a composition comprising the compounds of the invention for treating cancers; for inhibiting virus; for preventing cerebral aging; for improving memory; improving cerebral functions, for curing enuresis, frequent micturition, urinary incontinence, dementia, Alzheimer's disease, autism, brain trauma, Parkinson's disease or other diseases caused by cerebral dysfunctions; for treating arthritis, rheumatism, poor circulation, arteriosclerosis, Raynaud's syndrome, angina pectoris, cardiac disorder, coronary heart disease, headache, dizziness, kidney disorder; cerebrovascular diseasea; inhibiting NF-Kappa B activation; for treating brain edema, sever acute respiratory syndrome, respiratory viral diseases, chronic venous insufficiency, hypertension, chronic venous disease, anti-oedematous, anti inflammatory, haemonhoids, peripheral oedema formation, varicose vein disease, flu, post traumatic edema and postoperative swelling; for inhibiting ethanol absorption; for lowering blood sugar; for regulating the adreocorticotropin and corticosterone level; and for treating impotence or premature ejaculation or diabetes. See U.S. Ser. No. 10/906,303, filed Feb. 14, 2005, International Application No. PCT/US04/43465, filed Dec. 23, 2004, International Application No. PCT/US04/33359, filed Oct. 8, 2004, and U.S. Ser. No. 11/131,551, filed May 17, 2005, the contents of which are incorporated herein by reference.

This invention provides a composition for treating chronic venous insufficiency, peripheral edema, antilipemic, chronic venous disease, varicose vein disease, varicose syndrome, venous stasis, Expectorant, peripheral vascular disorders, cerebro-organic convulsion, cerebral circulation disorder, cerebral edema, psychoses, dysmenorrheal, hemorrhoids, episiotomies, haemonhoids, peripheral oedema formation or postoperative swelling; for reducing symptoms of leg pain; for treating pruritis, lower leg volume, thrombosis, thromophlebitis; for preventing gastric ulcers antispasmotic.

This invention provides a composition for AntiMS, antianeurysm, antiasthmatic, antibradykinic, anticapillarihemorrhagic, anticephalagic, anticervicobrachialgic, antieclamptic, antiedemic, antiencaphalitic, antiepigloftitic, antiexudative, antiflu, antifracture, antigingivitic, antihematomic, antiherpetic, antihistaminic, antihydrathritic, antimeningitic, antioxidant, antiperiodontic, antiphlebitic, antipleuritic, antiraucedo, antirhinitic, antitonsilitic, antiulcer, antivaricose, antivertiginous, cancerostatic, corticosterogenic, diuretic, fungicide, hemolytic, hyaluronidase inhibitor, lymphagogue, natriuretic, pesticide, pituitary stimulant, thymolytic, vasoprotective, venotonic treatment,

In an embodiment, an angeloyl group combined with a coumarin shows strong anti-tumor activities.

This invention provides a compound capable of reducing or inhibiting cancer cell growth, comprising the following structure:

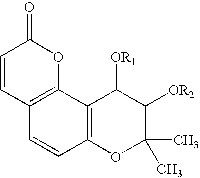

Wherein the R1, R2=Angeloyl, or tigloyl, or senecioyl, or acetyl group.

If the Angeloyl or tigloyl or senecioyl or acetyl in the above compound is replaced with hydroxyl group, the anti-tumor activities will be lost. The replacement of Angeloyl group with tigloyl group also reduces the anti-tumor activities. If we replace the acetyl group with Angeloyl group, the anti-tumor activities is increased.

In an embodiment, Angeloyl group combined with a coumarin shows activities. The structure is shown below:

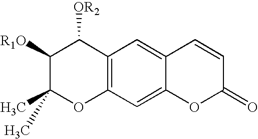

Wherein the R1, R2=Angeloyl or tigloyl or senecioyl or acetyl group.

The structure of the active compounds isolated from Angelica edulis Miyabe is shown below:

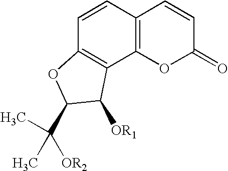

Wherein the R1, R2=Angeloyl or tigloyl or senecioyl or actyl group.

The above described compounds can be used for inhibiting cancer, wherein the cancer is not limited to breast cancer, leukocyte cancer, liver cancer, ovarian cancer, bladder cancer, prostate cancer, bone cancer, skin cancer, lung cancer, brain cancer, cervix cancer, KB cancer or brain cancer.

### Experimental Details

Experiment 1: Herb Extraction

(a) extracting powder of husks or branches or stems or leaves or kernels or roots or barks with organic solvent at ratio of 1:2 for 4-5 times for 20-35 hours each time to form an organic extract; (b) collecting the organic extract; (c) refluxing the organic extract for 2-3 times at 80° C. to form second extract; (d) removing the organic solvent from the second extract; and (e) drying and sterilizing the second extract to form the extract powder.

Experiment 2: Analysis of Extract Components by HPLC Chromatography

Methods

HPLC. A C-18 reverse phase μbondapak column (Water P/N 27324) was equilibrated with 10% acetonitrile, 0.005% Trifluoroacetic acid (equilibration solution). An extract of plants prepared using the methods described in Experiment 1 was dissolved in equilibration solution (1 mg/ml) before applying into the column. 20 ug of samples was applied into column. Elution conditions: Fractions were eluted (with flow rate 0.5 ml/min.) with acetonitrile gradient from 10% to 80% in 70 min, and then remains at 80% for 10 min. The acetonitrile concentration then decreased to 10% and remained at 10% for 25 min. The fractions were monitored at 207 nm and recorded in chart with a chart speed of 0.25 cm/min and with OD full scale of 0.128. Instruments. Waters Model 510 Solvent Delivery System; Waters 484 tunable Absorbance Detector; Waters 745/745B Data Module.

Absorbance analysis. The absorption profile of extract at various wavelengths was determined. An extract of the present invention was dissolved in 10% acetonitrile/TFA and scanned at 200-700 nm with a spectrophotometer [Spectronic Ins. Model Gene Sys2].

Results

HPLC. The peaks can be accounted for in the profile. The major peaks are labelled following increased concentration of acetonitrile elution.

Absorption maximum. Three absorption maximum were identified for plant extract; 207 nm, 278 nm and 500 nm.

Experiment 3: Determination of the Cell-growth Activity Effected by Extract with Cancer Cells Derived from Different Human Organs using MTT Assay

Methods and Materials

Cells. Human cancer cell lines were obtained from American Type Culture Collection: HTB-9 (bladder), HeLa-S3 (cervix), DU145 (prostate), H460 (lung), MCF-7 (breast), K562 (leukocytes), HCT116 (colon), HepG2 (liver), U2OS (bone), T98G (brain) and OVCAR-3 (ovary). Cells were grown in culture medium (HeLa-S3, DU145, MCF-7, Hep-G2 and T98G in MEN (Earle's salts); HTB-9, H460, K562, OVCAR-3 in RPMI-1640; HCT-116, U2OS in McCoy-5A) supplemented with 10% fetal calf serum, glutamine and antibiotics in a 5% CO2 humidified incubator at 37° C.

MTT assay. The procedure for MTT assay followed the method described in (Carmichael et al., 1987) with only minor modifications. Cells were seeded into a 96-wells plate at concentrations of 10,000/well (HTB-9, HeLa, H460, HCT116, T98G, OVCAR-3), 15,000/well (DU145, MCF-7, HepG2, U2OS), or 40,000/well (K562), for 24 hours before drug-treatment. Cells were then exposed to drugs for 48 hours (72 hours for HepG2, U2OS, and 96 hours for MCF-7). After the drug-treatment, MTT (0.5 mg/ml) was added to cultures for an hour. The formation of formazan (product of the reduction of tetrazolium by viable cells) was dissolved with DMSO and the O.D. at 490 nm was measured by an ELISA reader [Dynatech. Model MR700]. The MTT level of cells before drug-treatment was also measured (T0). The % cell-growth (% G) is calculated as:
% G=(TD−T0/TC−T0)×100 (1)
where TC or TD represent O.D. readings of control or drug-treated cells. When T0>TD, then the cytotoxicity (LC) expressed as % of the control is calculated as:
% LC=(TD−T0/T0)×100. (2)
Results

Among the 11 cell lines studies, inhibition of cell-grwoth after exposure of plant extract was observed. However, their sensitivity toward the extract is different. The response of the cell lines to the tested extract can be categorized into four groups: Most sensitive, Sensitive; Semi-sensitive; and least sensitive.

To investigate the inhibition components of the plant extract, the plant extract was fractionated.

Experiment 4: Purification of the Inhibition Components in Plant Extract.

(A) Fractionation of plant extracts with FPLC

Methods

Column. Octadecyl functionalized silica gel. Column dimension: 2 cm×28 cm; equilibrated with 10% acetonitrile—0.005% TFA before use.

Sample loading: 1-2 ml, concentration: 100 mg/ml in 10% acetonitrile/TFA.

Gradient elution condition: 10-80% acetonitrile in a total volume of 500 ml.

Monitor absorption wavelength: at 254 nm.

Fraction Collector: 5 ml/fractions (collect from 10% to 72% acetonitrile)

Instrument: AKTA-FPLC, P920 pump; Monitor UPC-900; Frac-900.

(B) Isolation of Component Ys with Preparative HPLC

Methods

Column: A preparative HPLC column (Waters Delta Pak C18-300A);

Elution conditions: 45% acetonitrile isocratic elution with flow rate of 1 ml/min.

Fractions are monitored at 207 nm and were collected and lyophilized.

Experiment 5: Determination of the Chemical Structure

Methods

NMR analysis. The pure compound Y of Xanthoceras sorbifolia was dissolved in pyridine-D5 with 0.05% v/v TMS. All NMR spectra were acquired using a Bruker Avance 600 MHz NMR spectrometer with a QXI probe (1H/13C/15N/31 P) at 298 K. The numbers of scans for 1D 1H spectra were 16 to 128, depending on the sample concentration. 2D HMQC spectra were recorded with spectral widths of 6000×24,000 Hz and data points of 2024×256 for t2 and t1 dimensions, respectively. The number of scans was 4 to 128. 2D HMBC were acquired with spectral widths of 6000×30,000 Hz and data points of 2024×512 for t2 and t1 dimensions, respectively. The number of scans was 64. The 2D data were zero-filled in t1 dimension to double the data points, multiplied by cosine-square-bell window functions in both t1 and t2 dimensions, and Fourier-transformed using software XWIN-NMR. The final real matrix sizes of these 2D spectra are 2048×256 and 2048×512 data points (F2×F1) for HMQC and HMBC, respectively.

Mass spectral analysis. The mass of samples was analyzed by (A) MALDI-TOF Mass Spectrometry and by (B) ESI-MS Mass spectrometry. (A) Samples for MALDI-TOF were first dissolved in acetonitrile, and then mixed with the matrix CHCA, i.e., Alpha-cyano-4-hydroxycinnamic acid, 10 mg CHCA/mL in 50:50 water/acetonitrile and 0.1% TFA in final concentration. The molecular weight was determined by the high resolution mass spectroscope analysis with standards. (B) For ESI, the sample was analyzed with LCQ DECA XP Plus machine made by Thermo Finnigan. It is ionized with ESI source and the solvent for the compound is acetonitrile.

Experiment 6: Determination the Anti Virus Activities of Compound of this Invention

The major procedures for the determination of antivirus activity are:

A. Determine the production of HIV virus after a non-lethal dosage of compound is added to the viral culture system.

B. Determine the growth activity of HIV virus after contact to compound. The steps for these experiments are:

1. Pre-treat HIV virus with different dosages of test compound for variable length of time.

2. Remove compound Y from virus.

3. Mix treated virus with cells.

4. Measure Virus production.

5. Negative control:—no virus in cell.

6. Positive control—untreated virus mixed with cell.

Result: The virus growth is inhibited after treatments of compound of this invention.

1-131. (canceled)

132. A composition comprising a compound selected from compounds shown in FIGS. 1 to 27.

133. The composition of claim 132, wherein the compounds of FIG. 1-2 comprises 2 angeloyl groups; or wherein the compounds of FIGS. 3A and 3B any two of R1, R2 and R4=angeloyl group; or wherein the compounds of FIGS. 5A, 5B, 6A and 6B any two of R1, R2, R3 and R4=angeloyl group; or wherein the compounds of FIGS. 7A, 7B, 7C and 7D any two of R1, R2 and R3=angeloyl group; or wherein the compound of FIG. 8 any two of R1, R2 and R3=angeloyl group; or wherein the compounds of FIGS. 9A, 9B and 10, any two of R1, R2 and R3=angeloyl group; or wherein the compounds of FIGS. 11, 12 and 13, R1 and R2=angeloyl group; or wherein compound of FIG. 14 any two of R1, R2 and R3=angeloyl group; or wherein the compounds of FIGS. 15 and 16, any two of R1, R2 and R6=angeloyl group; or wherein the compounds of FIG. 17-27, further comprises two angeloyl groups.

134. A compound selected from a compound of formula (5): 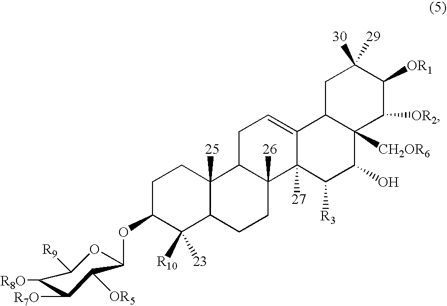
or a salt, ester, metabolite or derivative thereof, wherein R1 and R2 represent angeloyl group; R3 represents H or OH; R6 represents H or acetyl; R9 represents COOH or CH2OH; R10 represents CH3 or CH2OH; R5, R7 and R8 are H or/and sugar moiety, wherein the sugar moiety comprises at least one sugar, D-glucose, D-galactose, L-rhamnose, L-arabinose, D-xylose, alduronic acid, D-glucuronic acid, D-galacturonic acid, or derivative thereof, or a compound capable of performing the function of sugar moiety.

135. A compound of claim 134, wherein at least two of R1, R2, and R6 are angeloyl groups or a functional group capable of performing the function of angeloyl groups.

136. A compound of claim 134, wherein at least one of R1, R2, and R6 is a sugar moiety or rhamnose comprising at least two angeloyl groups, acetyl groups, tigloyl groups, senecioly groups, or an acid having two to five carbons or combination thereof.

137. A composition for inhibiting tumor cell growth, comprising the compound of claim 134.

138. A composition for reducing inflammation or for inhibiting virus or human immunodeficiency virus protease, comprising the compound of claim 134.

139. A composition for reducing inflammation or for inhibiting virus or human immunodeficiency virus protease, comprising the compound of claim 135.

140. A composition for reducing inflammation or for inhibiting virus or human immunodeficiency virus protease, comprising the compound of claim 136.

141. A composition for inhibiting cancer, comprising the compound of claim 134.

142. The composition of claim 141, wherein the cancer is breast cancer, leukocyte cancer, liver cancer, ovarian cancer, bladder cancer, prostate cancer, bone cancer, brain cancer, KB cancer or skin cancer.

143. A method for reducing leg swelling, reducing the symptom of chronic venous insufficiency, peripheral edema, antilipemic, chronic venous disease, varicose vein disease, varicose syndrome, venous stasis, Expectorant, peripheral vascular disorders, cerebro-organic convulsion, cerebral circulation disorder, cerebral edema, psychoses, dysmenorrheal, hemorrhoids, episiotomies, haemonhoids, peripheral oedema formation or postoperative swelling; for reducing symptoms of leg pain; for treating pruritis, lower leg volume, thrombosis, thromophlebitis; or for preventing gastric ulcers antispasmotic, or for AntiMS, antianeurysm, antiasthmatic, antibradykinic, anticapillarihemorrhagic, anticephalagic, anticervicobrachialgic, antieclamptic, antiedemic, antiencaphalitic, antiepiglottitic, antiexudative, antiflu, antifracture, antigingivitic, antihematomic, antiherpetic, antihistaminic, antihydrathritic, antimeningitic, antioxidant, antiperiodontic, antiphlebitic, antipleuritic, antiraucedo, antirhinitic, antitonsilitic, antiulcer, antivaricose, antivertiginous, cancerostatic, corticosterogenic, diuretic, fungicide, hemolytic, hyaluronidase inhibitor, lymphagogue, natriuretic, pesticide, pituitary stimulant, thymolytic, vasoprotective, venotonic treatment, comprising administering to a subject in need thereof, an effective amount of the composition of claim 132 to said subject.

144. A method for reducing leg swelling, reducing the symptom of chronic venous insufficiency, peripheral edema, antilipemic, chronic venous disease, varicose vein disease, varicose syndrome, venous stasis, Expectorant, peripheral vascular disorders, cerebro-organic convulsion, cerebral circulation disorder, cerebral edema, psychoses, dysmenorrheal, hemorrhoids, episiotomies, haemonhoids, peripheral oedema formation or postoperative swelling; for reducing symptoms of leg pain; for treating pruritis, lower leg volume, thrombosis, thromophlebitis; or for preventing gastric ulcers antispasmotic, or for AntiMS, antianeurysm, antiasthmatic, antibradykinic, anticapillarihemorrhagic, anticephalagic, anticervicobrachialgic, antieclamptic, antiedemic, antiencaphalitic, antiepiglottitic, antiexudative, antiflu, antifracture, antigingivitic, antihematomic, antiherpetic, antihistaminic, antihydrathritic, antimeningitic, antioxidant, antiperiodontic, antiphlebitic, antipleuritic, antiraucedo, antirhinitic, antitonsilitic, antiulcer, antivaricose, antivertiginous, cancerostatic, corticosterogenic, diuretic, fungicide, hemolytic, hyaluronidase inhibitor, lymphagogue, natriuretic, pesticide, pituitary stimulant, thymolytic, vasoprotective, venotonic treatment, comprising administering to a subject in need thereof, an effective amount of the composition of claim 134 to said subject.

145. A method for decreasing cholesterol or triglyceride level in the blood, for reducing urination, inhibiting cancer; for inhibiting virus; for preventing cerebral aging; for improving memory; improving cerebral functions, for inhibiting enuresis, frequent micturition, urinary incontinence, dementia, Alzheimer's disease, autism, brain trauma, Parkinson's disease or other diseases caused by cerebral dysfunctions; for treating arthritis, rheumatism, poor circulation, arteriosclerosis, Raynaud's syndrome, angina pectoris, cardiac disorder, coronary heart disease, headache, dizziness, kidney disorder; cerebrovascular disease; inhibiting NF-Kappa B activation; for treating brain edema, sever acute respiratory syndrome, respiratory viral diseases, chronic venous insufficiency, hypertension, chronic venous disease, anti-oedematous, anti inflammatory, haemonhoids, peripheral oedema formation, varicose vein disease, flu, post traumatic edema and postoperative swelling; for inhibiting ethanol absorption; for lowering blood sugar; for regulating the adreocorticotropin and corticosterone level; and for treating impotence or premature ejaculation or diabetes, comprising administering to a subject in need thereof, an effective amount of the composition of claim 132 to said subject.

146. A method for decreasing cholesterol or triglyceride level in the blood, for reducing urination, inhibiting cancer; for inhibiting virus; for preventing cerebral aging; for improving memory; improving cerebral functions, for inhibiting enuresis, frequent micturition, urinary incontinence, dementia, Alzheimer's disease, autism, brain trauma, Parkinson's disease or other diseases caused by cerebral dysfunctions; for treating arthritis, rheumatism, poor circulation, arteriosclerosis, Raynaud's syndrome, angina pectoris, cardiac disorder, coronary heart disease, headache, dizziness, kidney disorder; cerebrovascular disease; inhibiting NF-Kappa B activation; for treating brain edema, sever acute respiratory syndrome, respiratory viral diseases, chronic venous insufficiency, hypertension, chronic venous disease, anti-oedematous, anti inflammatory, haemonhoids, peripheral oedema formation, varicose vein disease, flu, post traumatic edema and postoperative swelling; for inhibiting ethanol absorption; for lowering blood sugar; for regulating the adreocorticotropin and corticosterone level; and for treating impotence or premature ejaculation or diabetes, comprising administering to a subject in need thereof, an effective amount of the composition of claim 134 to said subject.

147. A composition for inhibiting cancer, comprising the compound of claim 132.

148. A composition for inhibiting cancer, comprising the compound of claim 133.

149. The composition of claim 147, wherein the cancer is breast cancer, leukocyte cancer, liver cancer, ovarian cancer, bladder cancer, prostate cancer, bone cancer, brain cancer, KB cancer or skin cancer.

150. The composition of claim 148, wherein the cancer is breast cancer, leukocyte cancer, liver cancer, ovarian cancer, bladder cancer, prostate cancer, bone cancer, brain cancer, KB cancer or skin cancer.

151. A method for inhibiting tumor cell growth, human immunodeficiency virus (HIV), Severe Acute Respiratory Syndrome (SARS), flu disease, or viral activities, comprising contacting an effective amount of the compound shown in FIG. 17.
